(19) United States
(12) Patent Application Publication
(10) Pub. No.: US 2003/0059896 A1
(43) Pub. Date: Mar. 27, 2003

(54) Novel chlamydia antigens and corresponding DNA fragments

(75) Inventors: France Couture, St-David (CA); Josee Hamel, Sillery (CA); Bernard R. Brodeur, Sillery (CA); Denis Martin, St-Augustin-de-Desmaures (CA); Pascal Brassard, Plessisville (CA); Frederic Beaudoin, Ste-Foy (CA); Paul Prefontaine, Quebec (CA)

Correspondence Address: MILLEN, WHITE, ZELANO & BRANIGAN, P.C., 2200 CLARENDON BLVD., SUITE 1400, ARLINGTON, VA, 22201, US

(73) Assignee: Shire BioChem Inc., Laval (CA)

(21) Appl. No.: 10/022,832

(22) Filed: Dec. 20, 2001

Related U.S. Application Data
(60) Provisional application No. 60/256,941, filed on Dec. 21, 2000.

(51) Int. Cl. G01N 33/571; C07H 21/04; C12P 21/02; C12N 1/21; C07K 14/295; C12N 15/74

(52) U.S. Cl. 435/69.3; 435/252.3; 435/320.1; 435/7.36; 536/23.7; 530/350

(57) ABSTRACT

Chlamydia pneumoniae polypeptides and polynucleotides encoding them are disclosed. Said polypeptides are antigenic and therefore useful components for the prophylaxis, diagnosis or therapy of Chlamydial infection in animals. Also disclosed are recombinant methods of producing the protein antigens as well as diagnostic assays for detecting Chlamydia bacterial infection, particularly C. pneumoniae.

### FIELD OF THE INVENTION

The present invention is related to Chlamydia polypeptides and corresponding DNA fragments, which may be used to prevent, diagnose and/or treat Chlamydia infections.

### BACKGROUND OF THE INVENTION

Chlamydiae are obligate intracellular bacteria that cause a wide variety of diseases in man, animals and birds. Despite many differences (e.g. epidemiology, transmission routes, overall DNA homology, antigenic analysis), Chlamydiae share common biological and immunological features. The organism only grows into a specialized vacuole in the post-Golgi exocytic vesicular compartment of the eukaryotic cell. They undergo a distinct developmental cycle that alternates between an extracellular transmission cell, termed elementary body (EB) and an intracellular replicating cell, termed the reticulate body (RB). The organisms are capable of persisting in cells of an immune host presumably due to its evolved capabilities for immune evasion. Infection causes host cells to produce a variety of proinflammatory cytokines which likely contribute to disease pathogenesis (Rasmussen S. J., Eckmann L., Quayle A. J., Shen L., Zhang, Y. X., Anderson D. J., Fierer J., Stephens R. S. and Kagnoff M. F. (1997) Secretion of proinflammatory cytokines by epithelial cells in response to Chlamydia infection suggests a central role for epithelial cells in chlamydial pathogenesis. J. Clin. Invest. 99:77-87).

Four chlamydial species are currently known: Chlamydia trachomatis, Chlamydia pneumoniae, Chlamydia pecorum and Chlamydia psittaci. C. trachomatis and C. pneumoniae are two Chlamydia species that are natural human pathogen. C. psittaci strains are highly diverse, having been isolated from human and a very large number of avian and mammalian species. They have been grouped into eight biovars which produce a broad spectrum of diseases (Storz J., (1988), Overview of Animal Diseases Induced by Chlamydial Infections, In <<Microbiology of Chlamydia>> Ed. AL. Baron, CRC Press Inc. Boka Raton, Fla., pp. 167-192). C. pecorum has been isolated only from mammals.

In general, all chlamydiae appear to share a common immunobiology characterized by acute infection followed by immunity or by persistent or repeated infection that is associated with tissue damage and disease sequelae. Genome analysis shows that over 80% of C. pneumoniae and C. trachomatis protein-coding genes are orthologs that share a similar organization (Kalman S., Mitchell W., Marathe R., Lammel C., Fan J., Hyman R. W., Olinger L., Grinwood J., Davis R. W., Stephens R. S. (1999) Comparative genomes of Chlamydia pneumoniae and C. trachomatis. Nat Genet. 21:385-389). C. trachomatis causes blinding trachoma and is a major cause of sexually transmitted infections. C. pneumoniae infects the mucosal surfaces of respiratory tract causing sinusitis, pharyngitis, bronchitis and pneumonitis. As approximately, 10% of community-acquired pneumonia and 5% of bronchitis and sinusitis cases are caused by C. pneumoniae (Grayston, J. T. (2000) Background and current knowledge of Chlamydia pneumoniae and atherosclerosis. J. Infect. Dis. 181(Suppl 3):S402-410), prevention of them would already be beneficial. All the more that infection has been associated with other respiratory tract diseases, such as asthmatic bronchitis, adult-onset asthma, and chronic obstructive pulmonary diseases (COPD). Furthermore, numerous sero-epidemiological and pathological studies suggest that C. pneumoniae may disseminate from the respiratory tract to produce vascular infection and contribute to atherogenesis. Although this association has not been proven to be causal, it is becoming recognized as potentially highly important. In fact, C. pneumoniae is capable to surviving and replicating remarkably inside human mononuclear cells, a cell type that usually kills invading microbes. This probably provides them with a way to disseminate from the respiratory tract to elsewhere in the body, including artery walls (Gaydos C. A., Summersgill J. T., Sahney N. N., Ramirez J. A. and Quinn T. C. (1996) Replication of Chlamydia pneumoniae in vitro in human macrophages, endothelial cells and aortic artery smooth muscle cells. Infect. Immun. 64: 1614-1620). Consequently, infections may indirectly accelerate or enhance atherosclerosis in the presence of other risk factors by several mechanisms or they may induce atherosclerosis through local infection, inflammation or autoimmune reaction (Fong I. W. (2000) Emerging relations between infectious diseases and coronary artery disease and atherosclerosis. CMAJ 163:49-56).

From seroprevalence studies, C. pneumoniae infections usually happen between the ages of 5 and 20 years. In population, prevalence antibody rates reach 50% by age 20 and continue to rise in adults, reaching in old age about 80% in men and 70% in women (Wang S. P., and Grayston J. T. (1990) Population prevalence antibody to Chlamydia pneumoniae strain TWAR. In: Bowie W. R., Caldwell H. D., Jones R. P. et al., Eds. Chlamydial infections. Cambridge, UK: Cambridge University Press, pp 402-405; Wang S. P., and Grayston J. T. (1998) Chlamydia pneumoniae (TWAR) microimmunofluorescence studies—1998 update. In: Stephens R. S., Byrne B. I., Christiansen G. et al., Eds. Chlamydial infections. Berkely, Calif.: University of California, pp 155-158). Frequent reinfection and persistent chronic infection can best explain this prevalence. So, the great majority of the adult population has antibodies to C. pneumoniae, indicating past infection. But, protection conferred by natural chlamydial infection, is usually incomplete, transient (3-5 years)(Padnode D., Wang S. P. and Grayston J. T. (1990) Persistence of Chlamydia pneumoniae, strain TWAR micro-immunofluorescent antibody. In: Bowie W. R., Caldwell H. D., Jones R. P. et al., Eds. Chlamydial infections. Cambridge, UK: Cambridge University Press, pp 406-409) and strain specific. Until now, all tentatives have failed to result in an effective vaccine for human use. Therefore, there remains an unmet need for Chlamydia antigens that may be used as vaccine components for the prophylaxis and/or therapy of Chlamydia infection.

### SUMMARY OF THE INVENTION

According to one aspect, the present invention provides an isolated polynucleotide encoding a polypeptide having at least 70% identity to a second polypeptide comprising a sequence chosen from SEQ ID Nos: 2, 4, 6, 8, 10, 12, 14, 16, 18, 20, 22, 24, 26, 28, 30, 32, 34, 36 and 38 or fragments or analogs thereof.

According to one aspect, the present invention relates to polypeptides which comprise an amino acid sequence chosen from SEQ ID Nos: 2, 4, 6, 8, 10, 12, 14, 16, 18, 20, 22, 24, 26, 28, 30, 32, 34, 36 and 38 or fragments or analogs thereof.

In other aspects, there are provided polypeptides encoded by polynucleotides of the invention, pharmaceutical compositions, vectors comprising polynucleotides of the invention operably linked to an expression control region, as well as host cells transfected with said vectors and processes for producing polypeptides comprising culturing said host cells under conditions suitable for expression.

### BRIEF DESCRIPTION OF THE DRAWINGS

FIG. 1 is the DNA sequence of BVH-CPN1 gene from C. pneumoniae strain CWL-029; SEQ ID NO: 1. The underlined portion of the sequence represents the region coding for the leader peptide.

FIG. 2 is the deduced amino acid sequence of the full-length BVH-CPN1 from C. pneumoniae strain CWL-029; SEQ ID NO: 2. The underlined portion of the sequence represents the leader peptide.

FIG. 3 is the DNA sequence of BVH-CPN2 gene from C. pneumoniae strain CWL-029; SEQ ID NO: 3.

FIG. 4 is the deduced amino acid sequence of the full-length BVH-CPN2 from C. pneumoniae strain CWL-029; SEQ ID NO: 4.

FIG. 5 is the DNA sequence of BVH-CPN3 gene from C. pneumoniae strain CWL-029; SEQ ID NO: 5.

FIG. 6 is the deduced amino acid sequence of the full-length BVH-CPN3 from C. pneumoniae strain CWL-029; SEQ ID NO: 6.

FIG. 7 is the DNA sequence of BVH-CPN4 gene from C. pneumoniae strain CWL-029; SEQ ID NO: 7.

FIG. 8 is the deduced amino acid sequence of the full-length BVH-CPN4 from C. pneumoniae strain CWL-029; SEQ ID NO: 8.

FIG. 9 is the DNA sequence of BVH-CPN5 gene from C. pneumoniae strain CWL-029; SEQ ID NO: 9. The underlined portion of the sequence represents the region coding for the leader peptide.

FIG. 10 is the deduced amino acid sequence of the full-length BVH-CPN5 from C. pneumoniae strain CWL-029; SEQ ID NO: 10. The underlined portion of the sequence represents the leader peptide.

FIG. 11 is the DNA sequence of BVH-CPN6 gene from C. pneumoniae strain CWL-029; SEQ ID NO: 11.

FIG. 12 is the deduced amino acid sequence of the full-length BVH-CPN6 from C. pneumoniae strain CWL-029; SEQ ID NO: 12.

FIG. 13 is the DNA sequence of BVH-CPN7 gene from C. pneumoniae strain CWL-029; SEQ ID NO: 13.

FIG. 14 is the deduced amino acid sequence of the full-length BVH-CPN7 from C. pneumoniae strain CWL-029; SEQ ID NO: 14.

FIG. 15 is the DNA sequence of BVH-CPN8 gene from C. pneumoniae strain CWL-029; SEQ ID NO: 15.

FIG. 16 is the deduced amino acid sequence of the full-length BVH-CPN8 from C. pneumoniae strain CWL-029; SEQ ID NO: 16.

FIG. 17 is the DNA sequence of BVH-CPN9 gene from C. pneumoniae strain CWL-029; SEQ ID NO: 17.

FIG. 18 is the deduced amino acid sequence of the full-length BVH-CPN9 from C. pneumoniae strain CWL-029; SEQ ID NO: 18.

FIG. 19 is the DNA sequence of BVH-CPN10 gene from C. pneumoniae strain CWL-029; SEQ ID NO: 19.

FIG. 20 is the deduced amino acid sequence of the full-length BVH-CPN10 from C. pneumoniae strain CWL-029; SEQ ID NO: 20.

FIG. 21 is the DNA sequence of BVH-CPN11 gene from C. pneumoniae strain CWL-029; SEQ ID NO: 21.

FIG. 22 is the deduced amino acid sequence of the full-length BVH-CPN11 from C. pneumoniae strain CWL-029; SEQ ID NO: 22.

FIG. 23 is the DNA sequence of BVH-CPN12 gene from C. pneumoniae strain CWL-029; SEQ ID NO: 23.

FIG. 24 is the deduced amino acid sequence of the full-length BVH-CPN12 from C. pneumoniae strain CWL-029; SEQ ID NO: 24.

FIG. 25 is the DNA sequence of BVH-CPN13 gene from C. pneumoniae strain CWL-029; SEQ ID NO: 25.

FIG. 26 is the deduced amino acid sequence of the full-length BVH-CPN13 from C. pneumoniae strain CWL-029; SEQ ID NO: 26.

FIG. 27 is the DNA sequence of BVH-CPN14 gene from C. pneumoniae strain CWL-029; SEQ ID NO: 27. The underlined portion of the sequence represents the region coding for the leader peptide.

FIG. 28 is the deduced amino acid sequence of the full-length BVH-CPN14 from C. pneumoniae strain CWL-029; SEQ ID NO: 28. The underlined portion of the sequence represents the leader peptide.

FIG. 29 is the DNA sequence of BVH-CPN15 gene from C. pneumoniae strain CWL-029; SEQ ID NO: 29.

FIG. 30 is the deduced amino acid sequence of the full-length BVH-CPN15 from C. pneumoniae strain CWL-029; SEQ ID NO: 30.

FIG. 31 is the DNA sequence of BVH-CPN16 gene from C. pneumoniae strain CWL-029; SEQ ID NO: 31. The underlined portion of the sequence represents the region coding for the leader peptide.

FIG. 32 is the deduced amino acid sequence of the full-length BVH-CPN16 from C. pneumoniae strain CWL-029; SEQ ID NO: 32. The underlined portion of the sequence represents the leader peptide.

FIG. 33 is the DNA sequence of BVH-CPN17 gene from C. pneumoniae strain CWL-029; SEQ ID NO: 33.

FIG. 34 is the deduced amino acid sequence of the full-length VH-CPN17 from C. pneumoniae strain CWL-029; SEQ ID NO: 34.

FIG. 35 is the DNA sequence of BVH-CPN18 gene from C. pneumoniae strain CWL-029; SEQ ID NO: 35.

FIG. 36 is the deduced amino acid sequence of the full-length VH-CPN18 from C. pneumoniae strain CWL-029; SEQ ID NO: 36.

FIG. 37 is the DNA sequence of BVH-CPN19 gene from C. pneumoniae strain CWL-029; SEQ ID NO: 37. The underlined portion of the sequence represents the region coding for the leader peptide.

FIG. 38 is the deduced amino acid sequence of the full-length BVH-CPN19 from C. pneumoniae strain CWL-029; SEQ ID NO: 38. The underlined portion of the sequence represents the leader peptide.

FIG. 39 having Panels A to C, shows the anti-BVH-CPN1 immune response following intranasal immunization of BVH-CPN1 polypeptide in presence of the choleric toxin (CT; 1 μg) (A), the E. coli labile toxin (LT; 1 μg)(B) and subcutaneous immunization in presence of QuilA (15 μg) (C).

FIG. 40 shows the protection of the BVH-CPN1 polypeptide in the mouse model of pulmonary infection following intranasal immunization with 20 μg of BVH-CPN1 in presence of choleric toxin (CT; 1 μg) and the E. coli heat labile toxin (LT; 1 μg) and subcutaneous immunization with 20 μg of BVH-CPN1 in presence of QuilA (15 μg). Data corresponding to animals immunized with adjuvant only were compared to those immunized with the antigen in combination with the adjuvant according to the unpaired t test with Welch's correction.

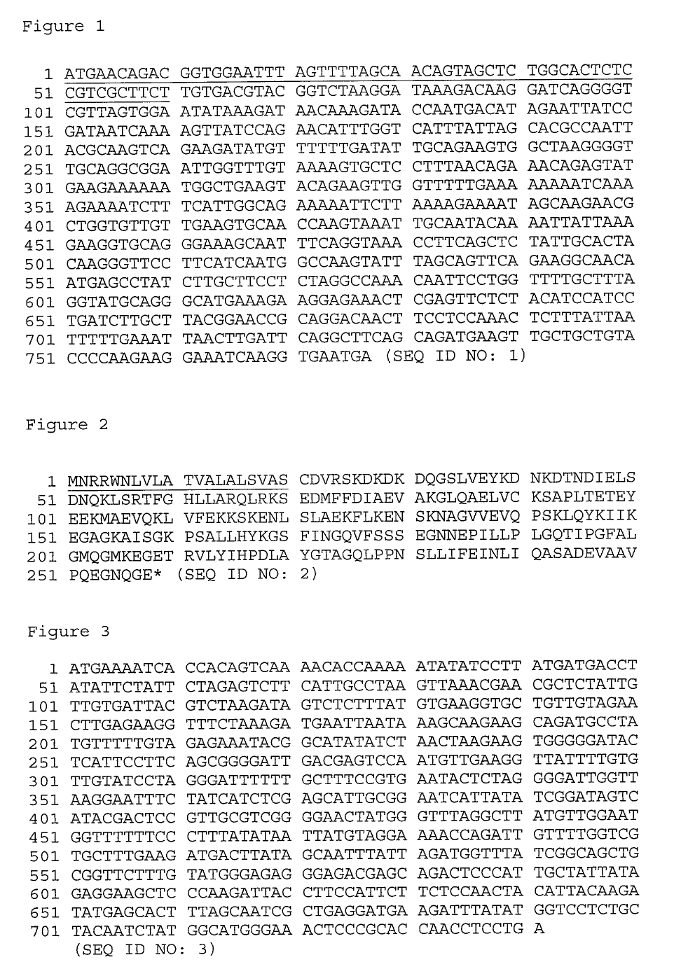

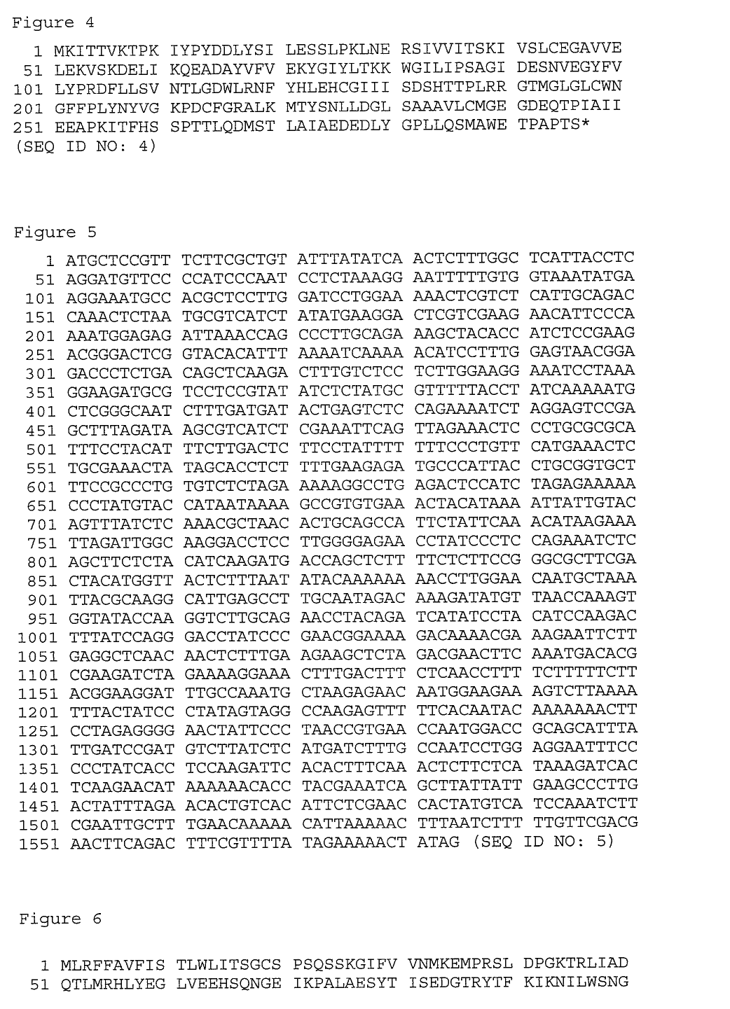

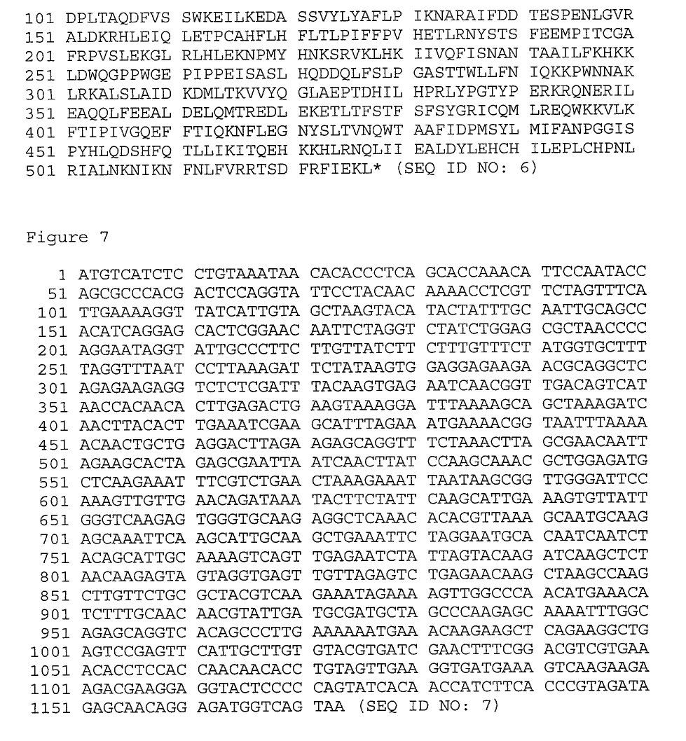

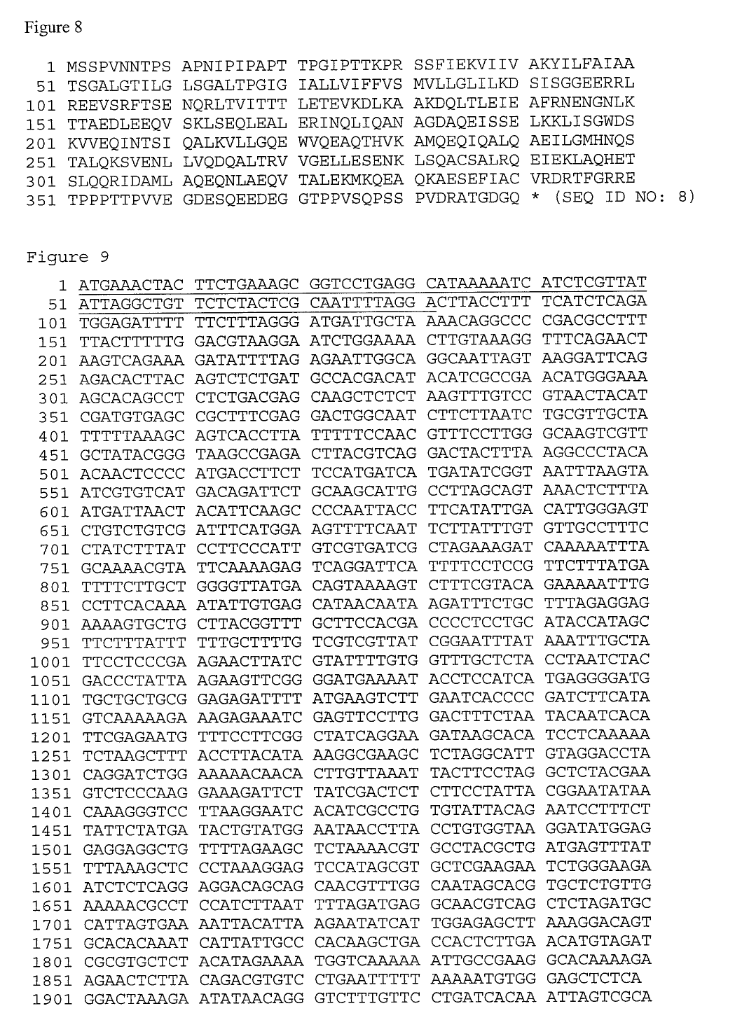

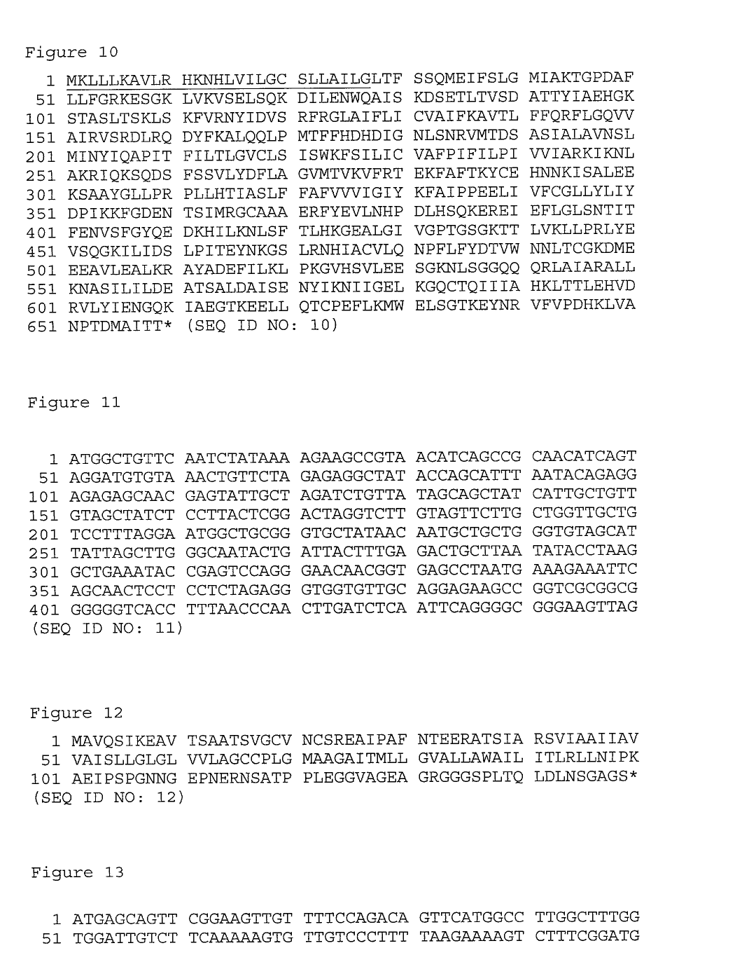

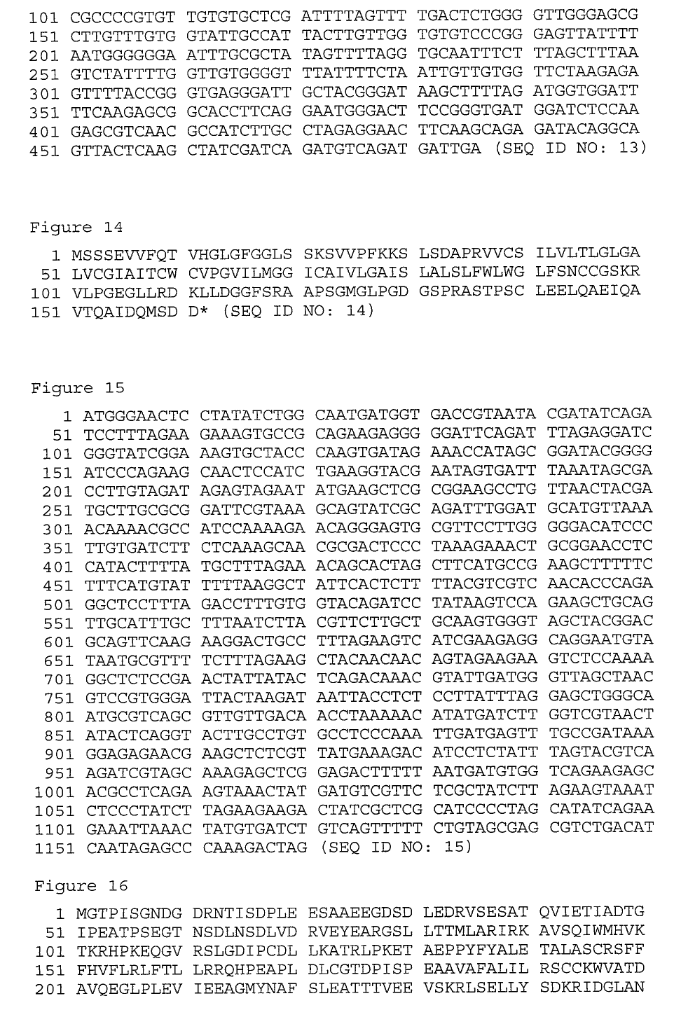

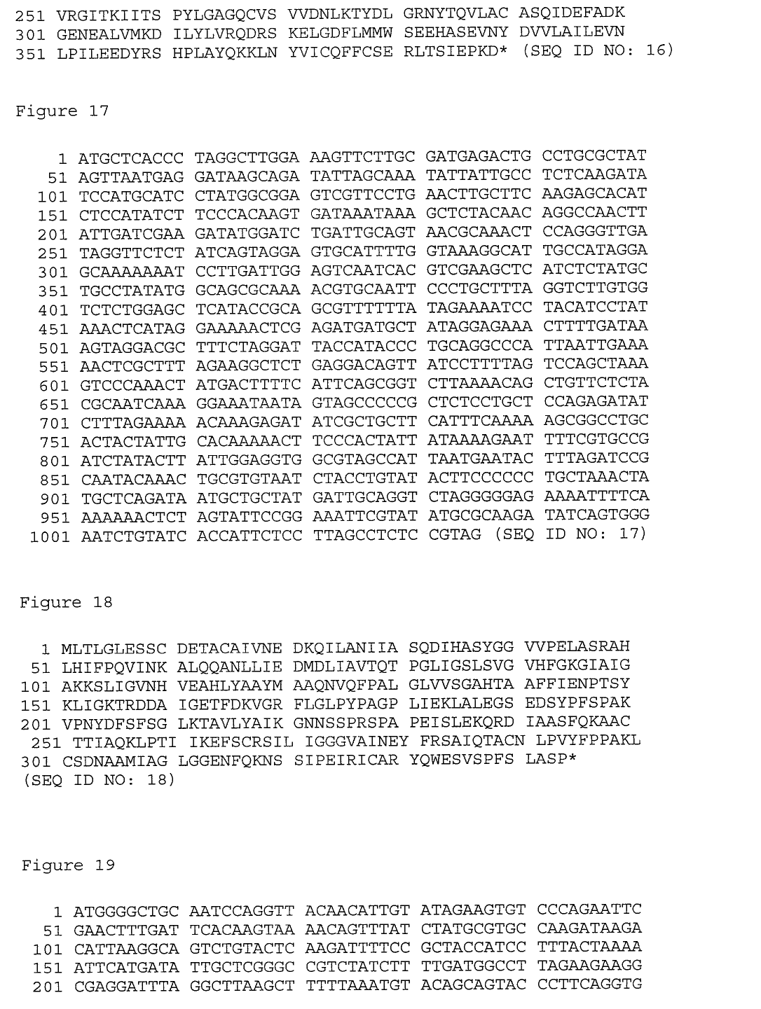

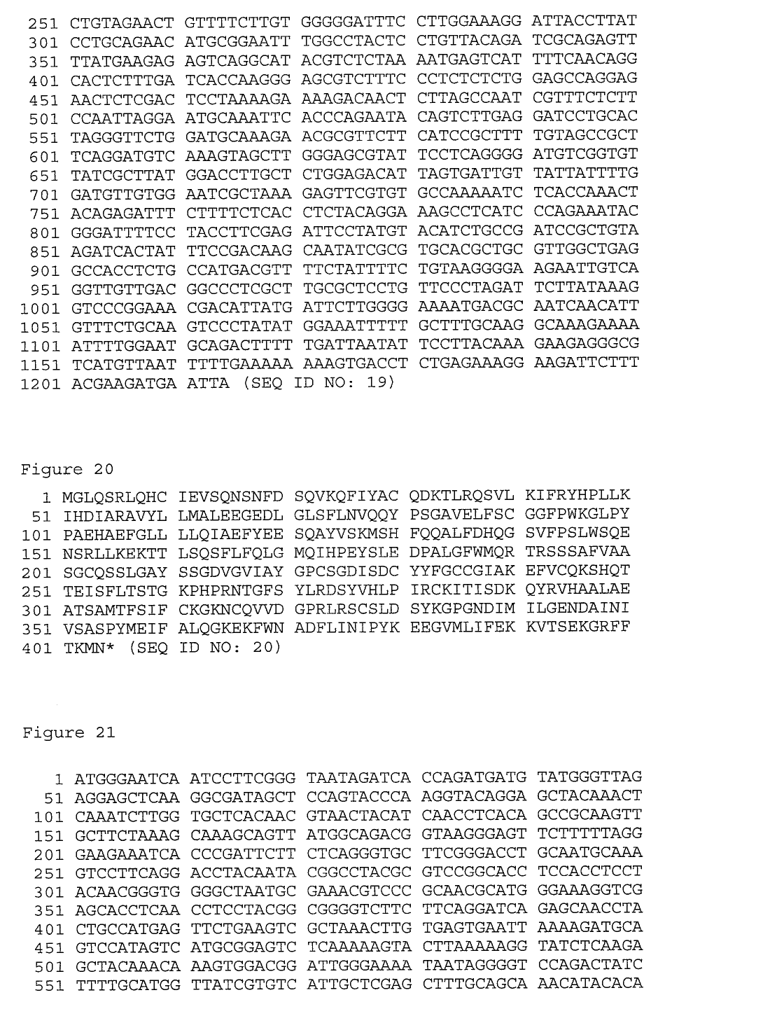

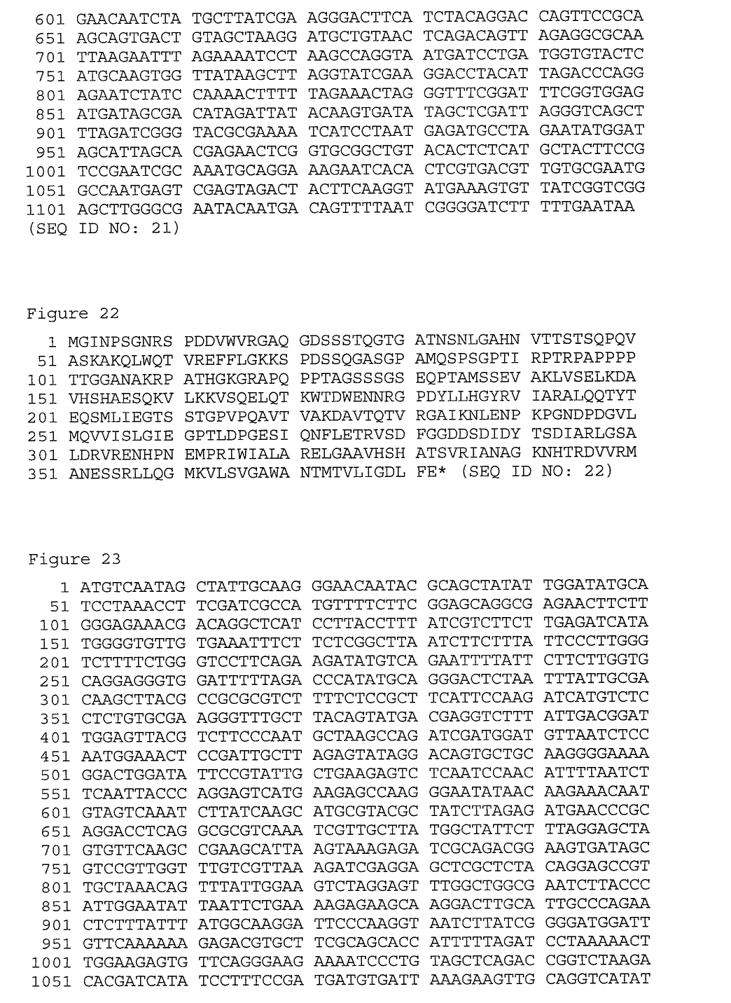

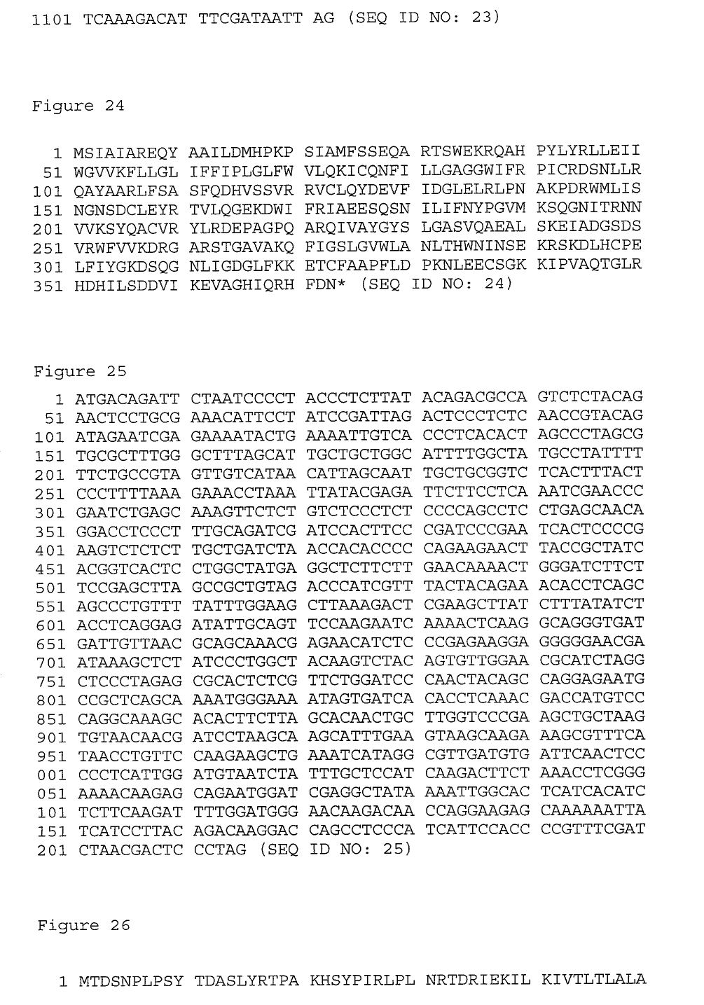

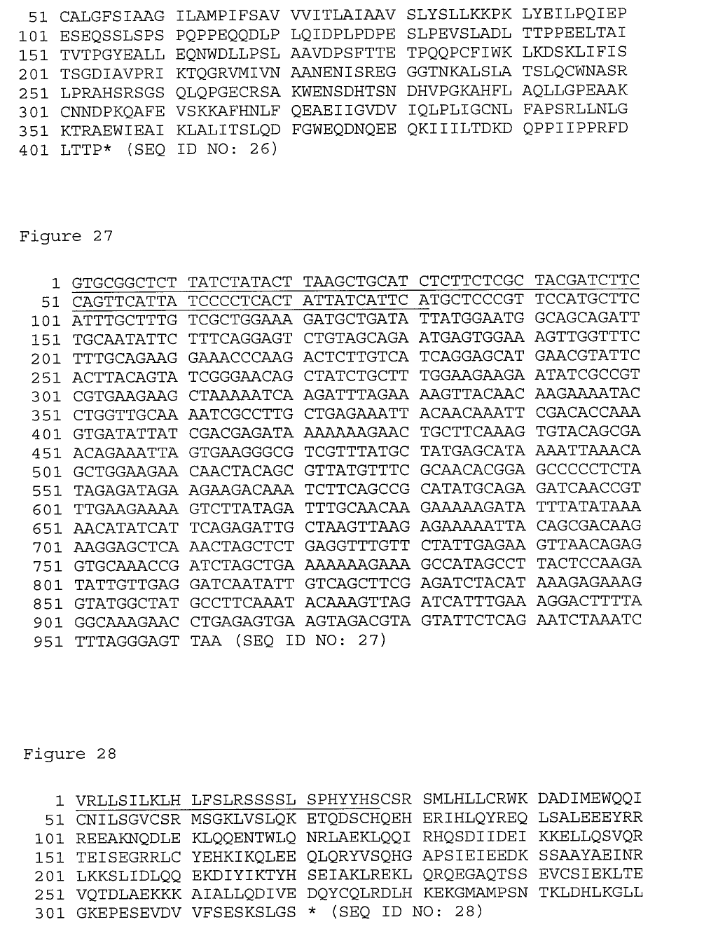

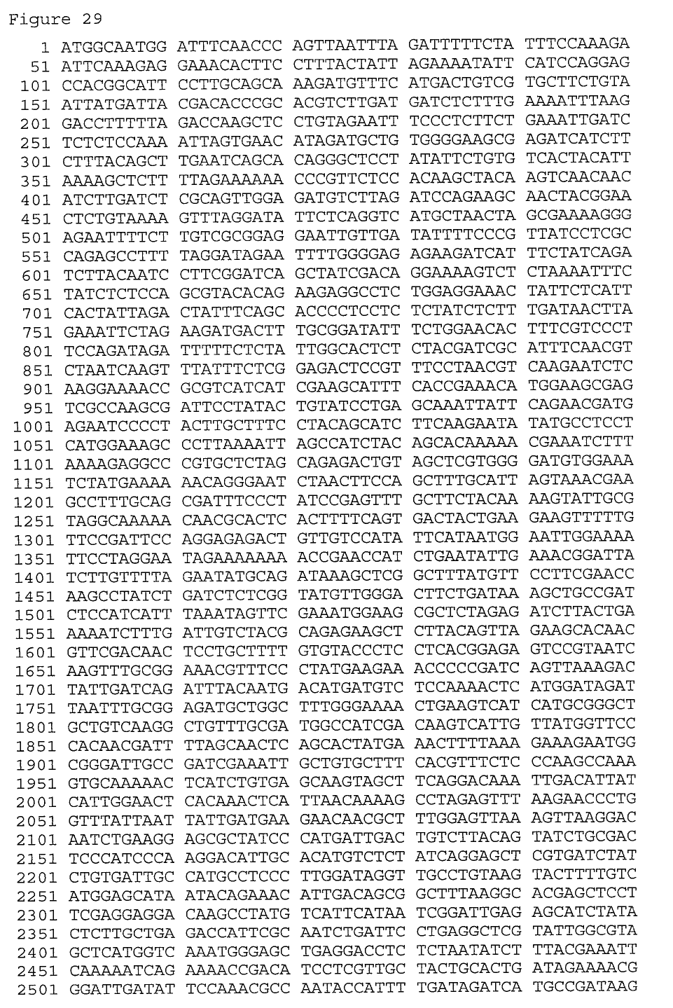

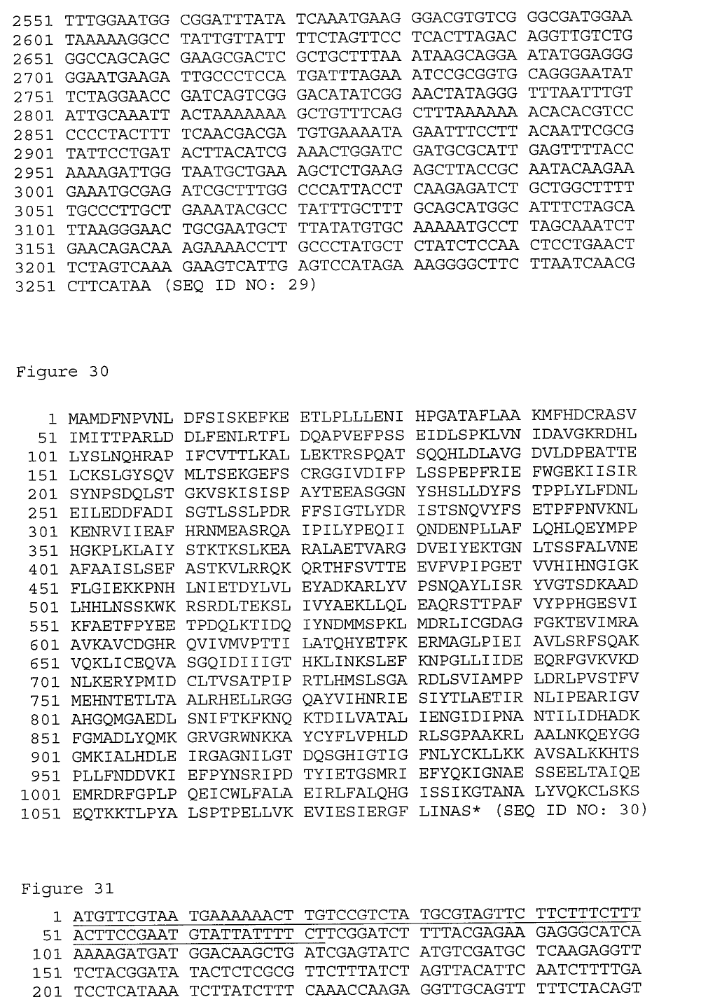

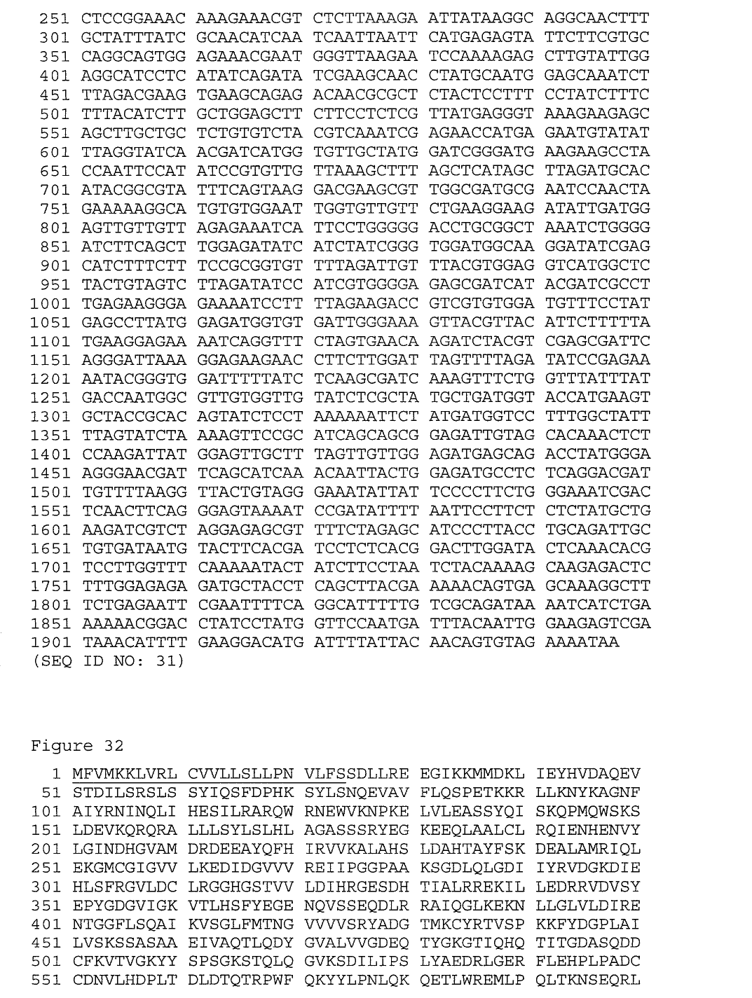

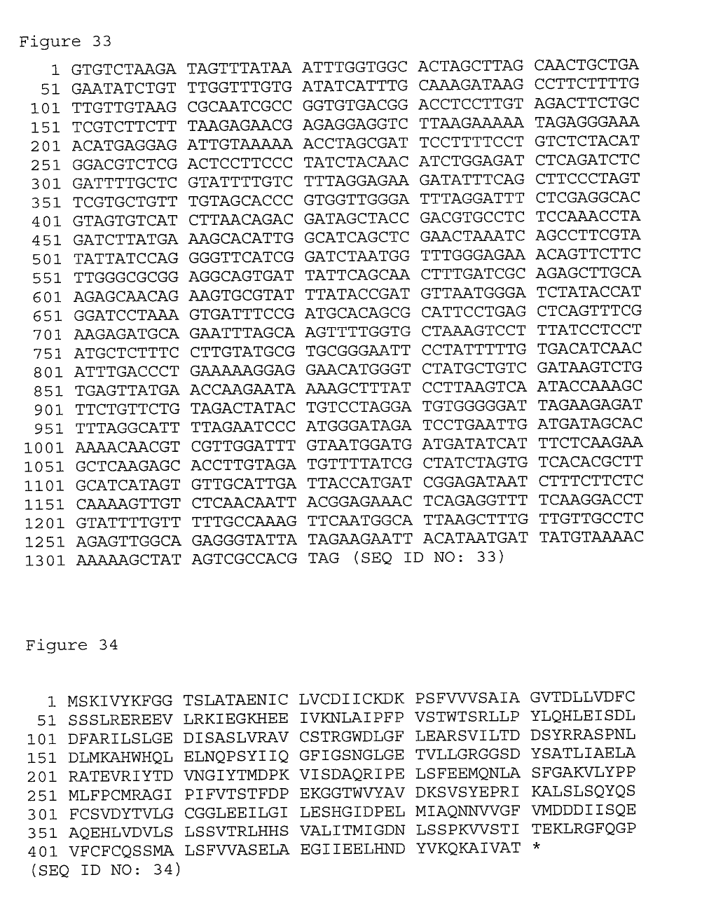

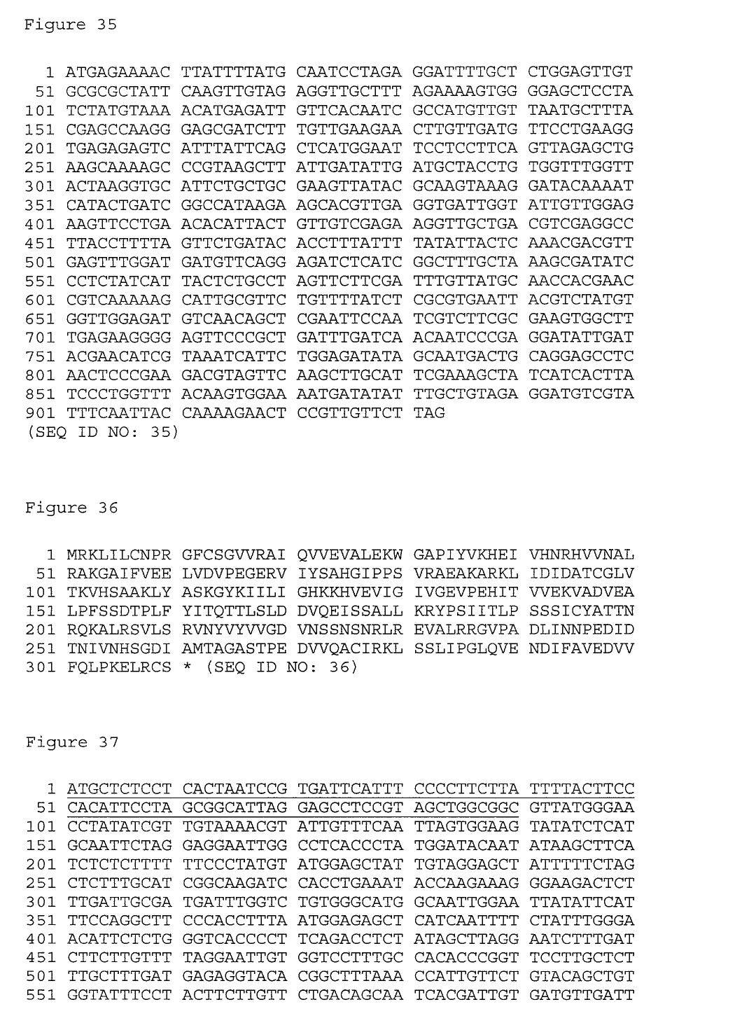

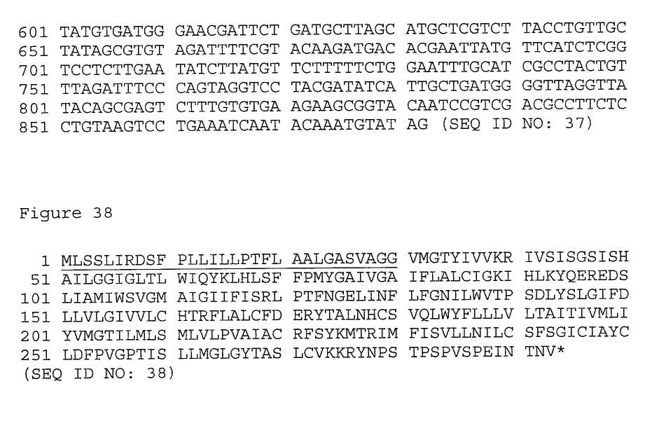

### DETAILED DESCRIPTION OF THE INVENTION

The present invention provides purified and isolated DNA molecules, which encode Chlamydial polypeptides that can be used to prevent, treat, and/or diagnose Chlamydial infection.

According to one aspect, the present invention provides an isolated polynucleotide encoding a polypeptide having at least 70% identity to a second polypeptide comprising a sequence chosen from SEQ ID Nos: 2, 4, 6, 8, 10, 12, 14, 16, 18, 20, 22, 24, 26, 28, 30, 32, 34, 36 and 38 or fragments or analogs thereof.

According to one aspect, the present invention provides an isolated polynucleotide encoding a polypeptide having at least 80% identity to a second polypeptide comprising a sequence chosen from SEQ ID Nos: 2, 4, 6, 8, 10, 12, 14, 16, 18, 20, 22, 24, 26, 28, 30, 32, 34, 36 and 38 or fragments or analogs thereof.

According to one aspect, the present invention provides an isolated polynucleotide encoding a polypeptide having at least 90% identity to a second polypeptide comprising a sequence chosen from SEQ ID Nos: 2, 4, 6, 8, 10, 12, 14, 16, 18, 20, 22, 24, 26, 28, 30, 32, 34, 36 and 38 or fragments or analogs thereof.

According to one aspect, the present invention provides an isolated polynucleotide encoding a polypeptide having at least 95% identity to a second polypeptide comprising a sequence chosen from SEQ ID Nos: 2, 4, 6, 8, 10, 12, 14, 16, 18, 20, 22, 24, 26, 28, 30, 32, 34, 36 and 38 or fragments or analogs thereof.

According to one aspect, the present invention provides a polynucleotide encoding a polypeptide comprising a sequence chosen from: SEQ ID NOs: 2, 4, 6, 8, 10, 12, 14, 16, 18, 20, 22, 24, 26, 28, 30, 32, 34, 36 and 38 or fragments or analogs thereof.

According to one aspect, the present invention provides a polynucleotide encoding a polypeptide capable of generating antibodies having binding specificity for a polypeptide having a sequence chosen from SEQ ID NOs: 2, 4, 6, 8, 10, 12, 14, 16, 18, 20, 22, 24, 26, 28, 30, 32, 34, 36 and 38 or fragments or analogs thereof.

According to one aspect, the present invention relates to polynucleotides encoding an epitope bearing portion of a polypeptide having a sequence chosen from SEQ ID NOS: 2, 4, 6, 8, 10, 12, 14, 16, 18, 20, 22, 24, 26, 28, 30, 32, 34, 36 and 38 or fragments or analogs thereof.

According to one aspect, the present invention relates to epitope bearing portions of a polypeptide having a sequence chosen from SEQ ID NOS: 2, 4, 6, 8, 10, 12, 14, 16, 18, 20, 22, 24, 26, 28, 30, 32, 34, 36 and 38 or fragments or analogs thereof.

According to one aspect, the present invention provides an isolated polynucleotide encoding a polypeptide having at least 70% identity to a second polypeptide comprising a sequence chosen from SEQ ID Nos: 2, 4, 6, 8, 10, 12, 14, 16, 18, 20, 22, 24, 26, 28, 30, 32, 34, 36 and 38.

According to one aspect, the present invention provides an isolated polynucleotide encoding a polypeptide having at least 80% identity to a second polypeptide comprising a sequence chosen from SEQ ID Nos: 2, 4, 6, 8, 10, 12, 14, 16, 18, 20, 22, 24, 26, 28, 30, 32, 34, 36 and 38.

According to one aspect, the present invention provides an isolated polynucleotide encoding a polypeptide having at least 90% identity to a second polypeptide comprising a sequence chosen from SEQ ID Nos: 2, 4, 6, 8, 10, 12, 14, 16, 18, 20, 22, 24, 26, 35 28, 30, 32, 34, 36 and 38.

According to one aspect, the present invention provides an isolated polynucleotide encoding a polypeptide having at least 95% identity to a second polypeptide comprising a sequence chosen from SEQ ID Nos: 2, 4, 6, 8, 10, 12, 14, 16, 18, 20, 22, 24, 26, 28, 30, 32, 34, 36 and 38.

According to one aspect, the present invention provides a polynucleotide encoding a polypeptide capable of generating antibodies having binding specificity for a polypeptide having a sequence chosen from SEQ ID NOs: 2, 4, 6, 8, 10, 12, 14, 16, 18, 20, 22, 24, 26, 28, 30, 32, 34, 36 and 38.

According to one aspect, the present invention provides a polynucleotide encoding an epitope bearing portion of a polypeptide having a sequence chosen from: SEQ ID NOs: 2, 4, 6, 8, 10, 12, 14, 16, 18, 20, 22, 24, 26, 28, 30, 32, 34, 36 and 38.

According to one aspect, the present invention relates to polynucleotides encoding an epitope bearing portion of a polypeptide having a sequence chosen from SEQ ID NOS: 2, 4, 6, 8, 10, 12, 14, 16, 18, 20, 22, 24, 26, 28, 30, 32, 34, 36 and 38.

According to one aspect, the present invention relates to epitope bearing portions of a polypeptide having a sequence chosen from SEQ ID NOS: 2, 4, 6, 8, 10, 12, 14, 16, 18, 20, 22, 24, 26, 28, 30, 32, 34, 36 and 38.

In accordance with the present invention, all polynucleotides encoding polypeptides are within the scope of the present invention.

Those skilled in the art will appreciate that the invention includes DNA molecules that encode analogs such as mutants, variants, homologues and derivatives of such polypeptides, as described herein in the present patent application. The invention also includes RNA molecules corresponding to the DNA molecules of the invention. In addition to the DNA and RNA molecules, the invention includes the corresponding polypeptides and monospecific antibodies that specifically bind to such polypeptides.

According to one aspect, the present invention relates to polypeptides having at least 70% identity to a second polypeptide having an amino acid sequence chosen from: SEQ ID NOs: 2, 4, 6, 8, 10, 12, 14, 16, 18, 20, 22, 24, 26, 28, 30, 32, 34, 36, 38 or fragments or analogs thereof.

According to one aspect, the present invention relates to polypeptides having at least 95% identity to a second polypeptide having an amino acid sequence chosen from: SEQ ID NOs: 2, 4, 6, 8, 10, 12, 14, 16, 18, 20, 22, 24, 26, 28, 30, 32, 34, 36, 38 or fragments or analogs thereof.

According to one aspect, the present invention relates to polypeptides characterized by the amino acid sequence comprising sequences from SEQ ID Nos:2, 4, 6, 8, 10, 12, 14, 16, 18, 20, 22, 24, 26, 28, 30, 32, 34, 36 and 38 or fragments or analogs thereof.

According to one aspect, the present invention relates to polypeptides capable of generating antibodies having binding specificity for a polypeptide having a sequence chosen from SEQ ID NOs: 2, 4, 6, 8, 10, 12, 14, 16, 18, 20, 22, 24, 26, 28, 30, 32, 34, 36, 38 or fragments or analogs thereof.

According to one aspect, the present invention relates to epitope bearing portions of a polypeptide having a sequence chosen from SEQ ID NOs: 2, 4, 6, 8, 10, 12, 14, 16, 18, 20, 22, 24, 26, 28, 30, 32, 34, 36, 38 or fragments or analogs thereof.

According to one aspect, the present invention relates to polypeptides having at least 70% identity to a second polypeptide having an amino acid sequence chosen from: SEQ ID NOs: 2, 4, 6, 8, 10, 12, 14, 16, 18, 20, 22, 24, 26, 28, 30, 32, 34, 36 or 38.

According to one aspect, the present invention relates to polypeptides having at least 95% identity to a second polypeptide having an amino acid sequence chosen from: SEQ ID NOs: 2, 4, 6, 8, 10, 12, 14, 16, 18, 20, 22, 24, 26, 28, 30, 32, 34, 36 or 38.

According to one aspect, the present invention relates to polypeptides characterized by the amino acid sequence comprising sequences from SEQ ID Nos 2, 4, 6, 8, 10, 12, 14, 16, 18, 20, 22, 24, 26, 28, 30, 32, 34, 36 or 38.

According to one aspect, the present invention relates to polypeptides capable of generating antibodies having binding specificity for a polypeptide having a sequence chosen from SEQ ID NOs: 2, 4, 6, 8, 10, 12, 14, 16, 18, 20, 22, 24, 26, 28, 30, 32, 34, 36 or 38.

According to one aspect, the present invention relates to epitope bearing portions of a polypeptide having a sequence chosen from SEQ ID NOs: 2, 4, 6, 8, 10, 12, 14, 16, 18, 20, 22, 24, 26, 28, 30, 32, 34, 36, 38 or fragments or analogs thereof.

In a further embodiment, the polypeptides in accordance with the present invention are antigenic.

In a further embodiment, the polypeptides in accordance with the present invention are immunogenic.

In a further embodiment, the polypeptides in accordance with the present invention can elicit an immune response in a host.

In a further embodiment, the present invention also relates to polypeptides which are able to raise antibodies having binding specificity to the polypeptides of the present invention as defined above.

An antibody that <<has binding specificity>> is an antibody that recognizes and binds the selected polypeptide but which does not substantially recognize and bind other molecules in a sample, e.g., a biological sample. Specific binding can be measured using an ELISA assay in which the selected polypeptide is used as an antigen.

In accordance with the present invention, “protection” in the biological studies is defined by a significant increase in the survival curve, rate or period. Statistical analysis using the Log rank test to compare survival curves, and Fisher exact test to compare survival rates and numbers of days to death, respectively, might be useful to calculate P values and determine whether the difference between the two groups is statistically significant. P values of 0.05 are regarded as not significant.

In an additional aspect of the invention there are provided antigenic/immunogenic fragments of the polypeptides of the invention, or of analogs thereof.

The fragments of the present invention should include one or more such epitopic regions or be sufficiently similar to such regions to retain their antigenic/immunogenic properties. Thus, for fragments according to the present invention the degree of identity is perhaps irrelevant, since they may be 100% identical to a particular part of a polypeptide or analog thereof as described herein. The present invention further provides fragments having at least 10 contiguous amino acid residues from the polypeptide sequences of the present invention. In one embodiment, at least 15 contiguous amino acid residues. In one embodiment, at least 20 contiguous amino acid residues.

The skilled person will appreciate that analogs of the polypeptides of the invention will also find use in the context of the present invention, i.e. as antigenic/immunogenic material. Thus, for instance polypeptides which include one or more additions, deletions, substitutions or the like are encompassed by the present invention.

As used herein, “fragments”, “analogs” or “derivatives” of the polypeptides of the invention include those polypeptides in which one or more of the amino acid residues are substituted with a conserved or non-conserved amino acid residue (preferably conserved) and which may be natural or unnatural. In one embodiment, derivatives and analogs of polypeptides of the invention will have about 70% identity with those sequences illustrated in the figures or fragments thereof. That is, 70% of the residues are the same. In a further embodiment, polypeptides will have greater than 80% identity. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

In a further embodiment, polypeptides will have greater than 70% homology. In a further embodiment, polypeptides will have greater than 75% homology. In a further embodiment, polypeptides will have greater than 80% homology. In a further embodiment, polypeptides will have greater than 85% homology. In a further embodiment, polypeptides will have greater than 90% homology. In a further embodiment, polypeptides will have greater than 95% homology. In a further embodiment, polypeptides will have greater than 99% homology. In a further embodiment, derivatives and analogs of polypeptides of the invention will have less than about 20 amino acid residue substitutions, modifications or deletions and more preferably less than 10. Preferred substitutions are those known in the art as conserved i.e. the substituted residues share physical or chemical properties such as hydrophobicity, size, charge or functional groups.

These substitutions are those having a minimal influence on the secondary structure and hydropathic nature of the polypeptide. Preferred substitutions are those known in the art as conserved, i.e. the substituted residues share physical or chemical properties such as hydrophobicity, size, charge or functional groups. These include substitutions such as those described by Dayhoff, M. in Atlas of Protein Sequence and Structure 5, 1978 and by Argos, P. in EMBO J. 8, 779-785, 1989. For example, amino acids, either natural or unnatural, belonging to one of the following groups represent conservative changes:

ala, pro, gly, gln, asn, ser, thr, val;

cys, ser, tyr, thr;

val, ile, leu, met, ala, phe;

lys, arg, orn, his;

and phe, tyr, trp, his.

The preferred substitutions also include substitutions of D-enantiomers for the corresponding L-amino acids.

The percentage of homology is defined as the sum of the percentage of identity plus the percentage of similarity or conservation of amino acid type.

In one embodiment, analogs of polypeptides of the invention will have about 70% identity with those sequences illustrated in the figures or fragments thereof. That is, 70% of the residues are the same. In a further embodiment, polypeptides will have greater than 80% homology. In a further embodiment, polypeptides will have greater than 90% homology. In a further embodiment, polypeptides will have greater than 95% homology. In a further embodiment, polypeptides will have greater than 99% homology. In a further embodiment, analogs of polypeptides of the invention will have less than about 20 amino acid residue substitutions, modifications or deletions and more preferably less than 10.

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

In an alternative approach, the analogs could be fusion polypeptides, incorporating moieties which render purification easier, for example by effectively tagging the desired polypeptide. It may be necessary to remove the “tag” or it may be the case that the fusion polypeptide itself retains sufficient antigenicity to be useful.

Thus, what is important for analogs, derivatives and fragments is that they possess at least a degree of the antigenicity/immunogenic of the protein or polypeptide from which they are derived.

Also included are polypeptides which have fused thereto other compounds which alter the biological or pharmacological properties of the polypeptide, i.e., polyethylene glycol (PEG) to increase half-life, leader or secretory amino acid sequences for ease of purification, prepro- and pro- sequences and polysaccharides.

Furthermore, in those situations where amino acid regions are found to be polymorphic, it may be desirable to vary one or more particular amino acids to more effectively mimic the different epitopes of the different chlamydial strains.

Moreover, the polypeptides of the present invention can be modified by terminal —NH₂ acylation (e.g. by acetylation or thioglycolic acid amidation, terminal carboxy amidation, e.g. with ammonia or methylamine) to provide stability, increased hydrophobicity for linking or binding to a support or other molecule.

Also contemplated are hetero and homo polypeptide multimers of the polypeptide fragments and analogs. These polymeric forms include, for example, one or more polypeptides that have been cross-linked with cross-linkers such as avidin/biotin, glutaraldehyde or dimethylsuperimidate. Such polymeric forms also include polypeptides containing two or more tandem or inverted contiguous sequences, produced from multicistronic mRNAs generated by recombinant DNA technology. In a further embodiment, the present invention also relates to chimeric polypeptides which comprise one or more polypeptides or fragments, analogs or derivatives thereof as defined in the figures of the present application.

In a further embodiment, the present invention also relates to chimeric polypeptides comprising two or more polypeptides having a sequence chosen from SEQ ID NOs: 2, 4, 6, 8, 10, 12, 14, 16, 18, 20, 22, 24, 26, 28, 30, 32, 34, 36, 38 or fragments or analogs thereof; provided that the polypeptides are linked as to formed a chimeric polypeptide.

In a further embodiment, the present invention also relates to chimeric polypeptides comprising two or more polypeptides having a sequence chosen from SEQ ID NOs: 2, 4, 6, 8, 10, 12, 14, 16, 18, 20, 22, 24, 26, 28, 30, 32, 34, 36 and 38, provided that the polypeptides are linked as to formed a chimeric polypeptide.

Preferably, a fragment, analog or derivative of a polypeptide of the invention will comprise at least one antigenic region i.e. at least one epitope.

In order to achieve the formation of antigenic polymers (i.e. synthetic multimers), polypeptides may be utilized having bishaloacetyl groups, nitroarylhalides, or the like, where the reagents being specific for thio groups. Therefore, the link between two mercapto groups of the different peptides may be a single bound or may be composed of a linking group of at least two, typically at least four and not more than 16, but usually not more than about 14 carbon atoms.

In a particular embodiment, polypeptide fragments or analogs of the invention do not contain a starting residue, such as methionine (Met) or Valine (Val).

Preferably, polypeptides will not incorporate a leader or secretory sequence (signal sequence) . The signal portion of a polypeptide of the invention may be determined according to established molecular biological techniques. The polypeptide of interest may be isolated from Chlamydial culture and subsequently sequenced to determine the initial residue of the mature protein and therefore the sequence of the mature polypeptide.

It is understood that polypeptides can be produced and/or used without their start codon (methionine or valine) and/or without their leader peptide to favor production and purification of recombinant polypeptides. It is known that cloning genes without sequences encoding leader peptides will restrict the polypeptides to the cytoplasm of E. coli and will facilitate their recovery (Glick, B. R. and Pasternak, J. J. (1998) Manipulation of gene expression in prokaryotes. In “Molecular biotechnology: Principles and applications of recombinant DNA”, 2nd edition, ASM Press, Washington D.C., p.109-143).

The polypeptides may be expressed with or without a leader or secretion sequence. In the former case, the leader may be removed using post-translational processing (see U.S. Pat. No. 4,431,739, U.S. Pat. No. 4,425, 437 and U.S. Pat. No. 4,338,397 incorporated herein by reference) or be chemically removed subsequent to purifying the expressed polypeptide.

According to another aspect of the invention, there are also provided (i) a composition of matter containing a polypeptide of the invention, together with a carrier, diluent or adjuvant; (ii) a pharmaceutical composition comprising a polypeptide of the invention and a carrier, diluent or adjuvant; (iii) a vaccine comprising a polypeptide of the invention and a carrier, diluent or adjuvant; (iv) a method for inducing an immune response against Chlamydia, in a host, by administering to the host, an immunogenically effective amount of a polypeptide of the invention to elicit an immune response, e.g., a protective immune response to Chlamydia (e.g. C.pneumoniae, C.trachomatis, C.psitacci or C.pecorum) ; and particularly, (v) a method for preventing and/or treating a Chlamydia (e.g. C.pneumoniae, C.trachomatis, C.psitacci or C.pecorum) infection, by administering a prophylactic or therapeutic amount of a polypeptide of the invention to a host in need.

According to another aspect of the invention, there are also provided (i) a composition of matter containing a polynucleotide of the invention, together with a carrier, diluent or adjuvant; (ii) a pharmaceutical composition comprising a polynucleotide of the invention and a carrier, diluent or adjuvant; (iii) a method for inducing an immune response against Chlamydia, in a host, by administering to the host, an immunogenically effective amount of a polynucleotide of the invention to elicit an immune response, e.g., a protective immune response to Chlamydia (e.g. C.pneumoniae, C.trachomatis, C.psitacci or C.pecorum); and particularly, (iv) a method for preventing and/or treating a Chlamydia (e.g. C.pneumoniae, C.trachomatis, C.psitacci or C.pecorum) infection, by administering a prophylactic or therapeutic amount of a polynucleotide of the invention to a host in need.

Before immunization, the polypeptides of the invention can also be coupled or conjugated to carrier proteins such as tetanus toxin, diphtheria toxin, hepatitis B virus surface antigen, poliomyelitis virus VP1 antigen or any other viral or bacterial toxin or antigen or any suitable proteins to stimulate the development of a stronger immune response. This coupling or conjugation can be done chemically or genetically. A more detailed description of peptide-carrier conjugation is available in Van Regenmortel, M. H. V., Briand J. P., Muller S., Plaué S., <<Synthetic Polypeptides as antigens>> in Laboratory Techniques in Biochemistry and Molecular Biology, Vol.19 (ed.) Burdou, R. H. & Van Knippenberg P. H. (198S), Elsevier New York.

According to another aspect, there are provided pharmaceutical comprising one or more Chlamydial polypeptides of the invention in a mixture with a pharmaceutically acceptable carrier, diluent or adjuvant. Suitable adjuvants include (1) oil-in-water emulsion formulations such as MF59™, SAF™, Ribi™; (2) Freund's complete or incomplete adjuvant; (3) salts i.e. AlK(SO₄)₂, AlNa(SO₄)₂, AlNH₄(SO₄)₂, Al(OH)₃, AlPO₄, silica, kaolin; (4) saponin derivatives such as Stimulon™ or particles generated therefrom such as ISCOMs (immunostimulating complexes); (5) cytokines such as interleukins, interferons, macrophage colony stimulating factor (M-CSF), tumor necrosis factor (TNF) ; (6) other substances such as carbon polynucleotides i.e. poly IC and poly AU, detoxified cholera toxin (CTB)and E.coli heat labile toxin for induction of mucosal immunity. A more detailed description of adjuvant is available in a review by M. Z. I Khan et al. in Pharmaceutical Research, vol.11, No.1 (1994) pp2-11, and also in another review by Gupta et al., in Vaccine, Vol.13, No14, pp1263-1276 (1995) and in WO 99/24578, which are herein incorporated by reference. Preferred adjuvants include QuilA™, QS21™, Alhydrogel™ and Adjuphos™.

In a further embodiment, there is provided a method of manufacturing a pharmaceutical composition comprising admixing a polypeptide of the invention with a pharmaceutically acceptable diluent, excipient or adjuvant.

In a further aspect, the invention provides a method for prophylactic or therapeutic treatment of Chlamydial bacterial infection in a host susceptible to Chlamydiae infection comprising administering to a host a therapeutic or prophylactic amount of a composition of the invention.

In a further aspect, the invention provides a method for prophylactic or therapeutic treatment of Chlamydial bacterial infection wherein said infection causes sinusitis, pharyngitis, bronchitis, pneumonitis, asthmatic bronchitis adult-onset asthma, chronic obstructive pulmonary diseases (CDPD), atherogenesis or atherosclerosis.

Pharmaceutical compositions of the invention may be used for the treatment or prophylaxis of chlamydial infection and/or diseases and symptoms mediated by chlamydial infection, in particular Chlamydia trachomatis, Chlamydia pneumoniae, Chlamydia pecorum or Chlamydia psittaci.

In one embodiment, pharmaceutical compositions of the invention are used for the treatment or prophylaxis of Chlamydia pneumoniae infection and/or diseases and symptoms mediated by Chlamydia pneumoniae infection.

In one embodiment, pharmaceutical compositions of the invention are used for the treatment or prophylaxis of Chlamydia psittaci infections and/or diseases and symptoms mediated by Chlamydia psittaci infection.

In one embodiment, pharmaceutical compositions of the invention are used for the treatment or prophylaxis of Chlamydia trachomatis infection and/or diseases and symptoms mediated by Chlamydia trachomatis infection.

Pharmaceutical compositions of the invention may be administered parenterally by injection, rapid infusion, nasopharyngeal adsorption, dermoadsorption, bucal or oral.

In a particular embodiment, pharmaceutical compositions are administered to those hosts at risk of Chlamydia infection such as infants, elderly and immunocompromised hosts.

In a further aspect, the invention provides the use of pharmaceutical composition of the invention for the prophylactic or therapeutic treatment of Chlamydia bacterial infection in a host susceptible to Chlamydia infection comprising administering to said host a therapeutic or prophylactic amount of a composition of the invention.

According to a further aspect, the Chlamydia polypeptides of the invention may be used in a kit comprising the polypeptides of the invention for detection or diagnosis of Chlamydia infection.

As used in the present application, the term <<hosts>> include animals. In a further embodiment, the animal is a mammal or a bird. In a further embodiment, the mammal is a human.

Pharmaceutical compositions are preferably in unit dosage form of about 0.001 to 100 μg/kg (antigen/body weight) and more preferably 0.01 to 10 μg/kg and most preferably 0.1 to 1 μg/kg, 1 to 3 times with an interval of about 1 to 6 weeks between immunizations.

Pharmaceutical compositions are preferably in unit dosage form of about 0.1 μg to 10 mg and more preferably 1 μg to 1 mg and most preferably 10 to 100 μg 1 to 3 times with an interval of about 1 to 6 week intervals between immunizations.

In one embodiment, polynucleotides are those illustrated in SEQ ID Nos: 1, 3, 5, 7, 9, 11, 13, 15, 17, 19, 21, 23, 25, 27, 29, 31, 33, 35 and 37 which may include the open reading frames (ORF), encoding the polypeptides of the invention.

It will be appreciated that the polynucleotide sequences illustrated in the figures may be altered with degenerated codons yet still encode the polypeptide of the invention. Accordingly the present invention further provides polynucleotide herein above described (or the complement sequence thereof) having 50% identity between sequences. In one embodiment, at least 70% identity between sequences. In one embodiment, at least 75% identity between sequences. In one embodiment, at least 80% identity between sequences. In one embodiment, at least 85% identity between sequences. In one embodiment, at least 90% identity between sequences. In a further embodiment, polynucleotides are hybridizable under stringent conditions, i.e. having at least 95% identity. In a further embodiment, more than 97% identity.

Suitable stringent conditions for hybridation can be readily determined by one of skilled in the art (see for example Sambrook et al., (1989) Molecular cloning: A Laboratory Manual, 2^{nd} ed, Cold Spring Harbor, N.Y.; Current Protocols in Molecular Biology, (1999) Edited by Ausubel F. M. et al., John Wiley & Sons, Inc., N.Y.).

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises SEQ ID NO: 2, 4, 6, 8, 10, 12, 14, 16, 18, 20, 22, 24, 26, 28, 30, 32, 34, 36, 38 or fragments or analogs thereof.

In a further embodiment, the present invention provides polynucleotides that hybridize under stringent conditions to either

(c) a DNA sequence encoding a polypeptide or

(d) the complement of a DNA sequence encoding a polypeptide;

wherein said polypeptide comprises SEQ ID NO: 2, 4, 6, 8, 10, 12, 14, 16, 18, 20, 22, 24, 26, 28, 30, 32, 34, 36 or 38.

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises at least 10 contiguous amino acid residues from a polypeptide comprising SEQ ID NO: 2, 4, 6, 8, 10, 12, 14, 16, 18, 20, 22, 24, 26, 28, 30, 32, 34, 36, 38 or fragments or analogs thereof.

In a further embodiment, the present invention provides polynucleotides that hybridize under stringent conditions to either

(c) a DNA sequence encoding a polypeptide or

(d) the complement of a DNA sequence encoding a polypeptide;

wherein said polypeptide comprises at least 10 contiguous amino acid residues from a polypeptide comprising SEQ ID NO: 2, 4, 6, 8, 10, 12, 14, 16, 18, 20, 22, 24, 26, 28, 30, 32, 34, 36 or 38.

As will be readily appreciated by one skilled in the art, polynucleotides include both DNA and RNA.

The present invention also includes polynucleotides complementary to the polynucleotides described in the present application.

In a further aspect, polynucleotides encoding polypeptides of the invention, or fragments or analogs thereof, may be used in a DNA immunization method. That is, they can be incorporated into a vector which is replicable and expressible upon injection thereby producing the antigenic polypeptide in vivo. For example polynucleotides may be incorporated into a plasmid vector under the control of the CMV promoter which is functional in eukaryotic cells. Preferably, the vector is injected intramuscularly.

According to another aspect, there is provided a process for producing polypeptides of the invention by recombinant techniques by expressing a polynucleotide encoding said polypeptide in a host cell and recovering the expressed polypeptide product. Alternatively, the polypeptides can be produced according to established synthetic chemical techniques, i.e. solution phase or solid phase synthesis of oligopeptides which are ligated to produce the full polypeptide (block ligation).

General methods for obtention and evaluation of polynucleotides and polypeptides are described in the following references: Sambrook et al., (1989) Molecular cloning: A Laboratory Manual, 2^{nd} ed, Cold Spring Harbor, N.Y.; Current Protocols in Molecular Biology, (1999) Edited by Ausubel F. M. et al., John Wiley & Sons, Inc., N.Y.; PCR Cloning Protocols, from Molecular Cloning to Genetic Engineering, (1997) Edited by White B. A., Humana Press, Totowa, N.J., 490 pages; Protein Purification, Principles and Practices, (1993) Scopes R. K., Springer-Verlag, N.Y., 3^{rd} Edition, 380 pages; Current Protocols in Immunology, (1999) Edited by Coligan J. E. et al., John Wiley & Sons Inc., N.Y..

For recombinant production, host cells are transfected with vectors which encode the polypeptide, and then cultured in a nutrient media modified as appropriate for activating promoters, selecting transformants or amplifying the genes. Suitable vectors are those that are viable and replicable in the chosen host and include chromosomal, non-chromosomal and synthetic DNA sequences e.g. bacterial plasmids, phage DNA, baculovirus, yeast plasmids, vectors derived from combinations of plasmids and phage DNA. The polypeptide sequence may be incorporated in the vector at the appropriate site using restriction enzymes such that it is operably linked to an expression control region comprising a promoter, ribosome binding site (consensus region or Shine-Dalgarno sequence), and optionally an operator (control element) One can select individual components of the expression control region that are appropriate for a given host and vector according to established molecular biology principles (Sambrook et al., (1989) Molecular Cloning: A Laboratory manual, 2^{nd} ed., Cold Spring Harbor, N.Y.; Current Protocols in Molecular Biology, (1999) Edited by Ausubel F. M. et al., John Wikey & Sons, Inc., N.Y., incorporated herein by reference). Suitable promoters include but are not limited to LTR or SV40 promoter, E. coli lac, tac or trp promoters and the phage lambda PL promoter. Vectors will preferably incorporate an origin of replication as well as selection markers, i.e. antibiotic resistance gene. Suitable bacterial vectors include pET, pQE70, pQE60, pQE-9, pD10 phagescript, PSIX174, pBluescript SK, pbsks, pNH8A, pNH16A, pNH18A, pNH46A, ptrc99a, pKK223-3, pKK233-3, pDR540, pRIT5 and eukaryotic vectors pBlueBacIII, pWLNEO, pSV2CAT, pOG44, pXT1, pSG, pSVK3, PBPV, pMSG and PSVL. Host cells may be bacterial (i.e. E. coli, Bacillus subtilis, Streptomyces), fungal (i.e. Aspergillus niger, Aspergillus nidulins), yeast (i.e. Saccharomyces) or eukaryotic (i.e. CHO, COS).

Upon expression of the polypeptide in culture, cells are typically harvested by centrifugation then disrupted by physical or chemical means (if the expressed polypeptide is not secreted into the media) and the resulting crude extract retained to isolate the polypeptide of interest. Purification of the polypeptide from culture media or lysate may be achieved by established techniques depending on the properties of the polypeptide: ammonium sulfate or ethanol precipitation, acid extraction, anion or cation exchange chromatography, phosphocellulose chromatography hydrophobic interaction chromatography, hydroxylapatite chromatography and lectin chromatography. Final purification may be achieved using HPLC.

According to a further aspect, the Chlamydial polypeptides of the invention may be used in a diagnostic test for Chlamydial infection, in particular C. pneumoniae infection. Several diagnostic methods are possible, for example detecting Chlamydia organism in a biological sample, the following procedure may be followed:

a) obtaining a biological sample from a host;

b) incubating an antibody or fragment thereof reactive with a Chlamydia polypeptide of the invention with the biological sample to form a mixture, and

c) detecting specifically bound antibody or bound fragment in the mixture which indicates the presence of Chlamydia.

Alternatively, a method for the detection of antibody specific to a Chlamydial antigen in a biological sample containing or suspected of containing said antibody may be performed as follows:

a) obtaining a biological sample from a host;

b) incubating one or more Chlamydial polypeptides of the invention or fragments thereof with the biological sample to form a mixture; and

c) detecting specifically bound antigen or bound fragment in the mixture which indicates the presence of antibody specific to Chlamydia.

One of skill in the art will recognize that this diagnostic test may take several forms, including an immunological test such as an enzyme-linked immunoadsorbent assay (ELISA), a radioimmunoassay or a latex agglutination assay, essentially to determine whether antibodies specific for the protein are present in an organism.

The DNA sequences encoding polypeptides of the invention may also be used to design DNA probes for use in detecting the presence of Chlamydia in a biological sample suspected of containing such bacteria. The detection method of this invention comprises

a) obtaining the biological sample from a host;

b) incubating one or more DNA probes having a DNA sequence encoding a polypeptide of the invention or fragments thereof with the biological sample to form a mixture; and

c) detecting specifically bound DNA probe in the mixture which indicates the presence of Chlamydia bacteria.

The DNA probes of this invention may also be used for detecting circulating Chlamydia (i.e. Chlamydial nucleic acids)in a sample, for example using a polymerase chain reaction, as a method of diagnosing Chlamydia infections. The probe may be synthesized using conventional techniques and may be immobilized on a solid phase or may be labelled with a detectable label. A preferred DNA probe for this application is an oligomer having a sequence complementary to at least about 6 contiguous nucleotides of the C. pneumoniae polypeptides of the invention.

Another diagnostic method for the detection of Chlamydia in a host comprises:

a) labelling an antibody reactive with a polypeptide of the invention or fragment thereof with a detectable label;

b) administering the labelled antibody or labelled fragment to the host; and

c) detecting specifically bound labelled antibody or labelled fragment in the host which indicates the presence of Chlamydia.

A further aspect of the invention is the use of the Chlamydia polypeptides of the invention as immunogens for the production of specific antibodies for the diagnosis and in particular the treatment of Chlamydia infection. Suitable antibodies may be determined using appropriate screening methods, for example by measuring the ability of a particular antibody to passively protect against Chlamydia infection in a test model. One example of an animal model is the mouse model described in the example herein. The antibody may be a whole antibody or an antigen-binding fragment thereof and may belong to any immunoglobulin class. The antibody or fragment may be of animal origin, specifically of mammalian origin and more specifically of murine, rat or human origin. It may be a natural antibody or a fragment thereof, or if desired, a recombinant antibody or antibody fragment. The term recombinant antibody or antibody fragment means antibody or antibody fragment which was produced using molecular biology techniques. The antibody or antibody fragments may be polyclonal or preferably monoclonal. It may be specific for a number of epitopes associated with the C. pneumoniae polypeptides but is preferably specific for one.

A further aspect of the invention is the use of the antibodies directed to the polypeptides of the invention for passive immunization. One could use the antibodies described in the present application. Suitable antibodies may be determined using appropriate screening methods, for example by measuring the ability of a particular antibody to passively protect against chlamydia infection in a test model. One example of an animal model is the mouse model described in the examples herein. The antibody may be a whole antibody or an antigen-binding fragment thereof and may belong to any immunoglobulin class. The antibody or fragment may be of animal origin, specifically of mammalian origin and more specifically of murine, rat or human origin. It may be a natural antibody or a fragment thereof, or if desired, a recombinant antibody or antibody fragment. The term recombinant antibody or antibody fragment means antibody or antibody fragment which was produced using molecular biology techniques. The antibody or antibody fragments may be polyclonal, or preferably monoclonal. It may be specific for a number of epitopes associated with the chlamydia polypeptides but is preferably specific for one.

According to one aspect, the present invention provides the use of an antibody for treatment and/or prophylaxis of Chlamydial infections.

Unless otherwise defined, all technical and scientific terms used herein have the same meaning as commonly understood by one of ordinary skill in the art to which this invention belong. All publications, patent applications, patents and other references mentioned herein are incorporated by reference in their entirety. In case of conflict, the present specification including definitions will control. In addition, the materials, methods, and examples are illustrative only and not intended to be limiting.

### ISOLATION OF CHLAMYDIA POLYPEPTIDES AND IDENTIFICATION OF GENES ENCODING THESE POLYPEPTIDES

### ISOLATION OF INFECTION SPECIFIC CHLAMYDIA POLYPEPTIDES

Bacterial Strains

C. pneumoniae CWL-029 (American Culture Collection, Rockville, Md.) was cultivated in HEp-2 and McCoy cells (American Culture Collection, Rockville, Md.) using standard methods (Maass M. and Harig U. (1995) Evaluation of culture conditions used for isolation of Chlamydia pneumoniae. Clin. Microbiol. Infect. Dis. 103: 141-148; Roblin P. M., Dumornay W. and Hammerschlag M. R. (1992) Use of HEp-2 cells for improved isolation and passage of Chlamydia pneumoniae. J. Clin. Microbiol. 30: 1968-1971; Wong K. H., Skelton S. K. and Chan Y. K. (1992) Efficient culture of Chlamydia pneumoniae with cell lines derived from the human respiratory tract. J. Clin. Microbiol. 30:1625-1630). Purified chlamydiae were obtained using Hypaque-M 76% (diatrizoate meglumine and diatrizoate sodium (Sanofi Winthrop, Laval, Quebec) density gradient centrifugation (Caldwell H. D., Kromhout J., and Schachter J. (1981) Purification and partial characterization of the outer membrane protein of Chlamydia trachomatis. Infect. Immun. 31:1161-1176; Miyashita, M. and Matsumoto, A. (1992) Establishment of a particle-counting method for purified elementary bodies of Chlamydiae and evaluation of sensitivities of the IDEIA Chlamydia kit and DNA probe by using the purified elementary bodies. J. Clin. Microbiol. 30:2911-2916). Escherichia coli DH5α (GIBCO BRL, Burlington, Ontario) was used as the host for recombinant DNA. E. coli XL1-Blue MRF′ (Stratagene, LaJolla, Calif.) was used as the host strain for infection with lambda ZAP Express phage vector. E. coli XLOLR (Stratagene) was used as the host strain for infection with in vivo excised filamentous lambda ZAP Express.

Antisera

Rabbit and mice antisera against Chlamydia-infected McCoy cells were produced following three-week intervals subcutaneous immunization in presence of QuilA adjuvant (Cedarlane Laboratories Ltd, Hornby, Canada). Sera were collected 28 days after the tertiary immunization.

Four human antisera were selected on the basis of their reactivity by ELISA and Western blotting on heat-killed purified Chlamydiae.

C. pneumoniae library construction and screening C. pneumoniae CWL-029 DNA was extracted using the RNA/DNA Midi Kit (Qiagen). DNA was partially digested with Tsp509I and ligated to EcoRI digested X-ZAP Express phage arms (Stratagene). The ligation was packaged in vitro with Gigapack extracts according to the manufacturer's recommendations (Stratagene). Recombinant phage were plated on E. coli XL1-Blue MRF′ at density of 2.5×10⁴ PFU/150 mm (diameter) plate. Following eight hours incubation, the plates were overlaid with nitrocellulose disks and the resulting lifts were processed for immunoblotting with human and rabbit sera. Positive clones were picked and plaque-purified. Recombinant pBK-CMV plasmids coding for the chlamydial genes of interest were recovered from the purified bacteriophage using ExAssist filamentous helper phage and the lambda-resistant strain E. coli XLOLR by in vivo excision.

Identification of Antigens

SDS-PAGE and immunoblot analysis of lysates of these recombinant E. coli showed that each expressed one or more proteins that reacted with human or rabbit antisera.

DNA Sequencing and Sequence Analysis

The genomic clones were sequenced with the Taq DyeDeoxy Terminator Cycle Sequencing Kit (Applied Biosystem, Foster City, Calif.). Several internal primers were designed to sequence further into the cloned inserts. Sequence assembly was performed using Sequencher software (Gene Codes Corporation, Ann Arbor, Mich.) and sequence analysis was performed with McVector software (Oxford Molecular Ltd., Campbell, Calif.).

### EXAMPLE 1

This example illustrates the cloning of C. pneumoniae genes into expression vector

The coding region of C. pneumoniae genes BVH-CPNL to BVH-CPN19 (SEQ ID NO:1 to NO:19) were amplified by PCR (DNA Thermal Cycler GeneAmp PCR system 2400 Perkin Elmer, San Jose, Calif.) from genomic DNA of C. pneumoniae strain CWL-029 by using the following oligonucleotide primers that contained base extensions for the addition of restriction sites BamHI (GGATCC), NcoI (CCATGG), NdeI (CATATG), NotI (GCGGCCGC) or XhoI (CTCGAG) (Table 1). PCR products were purified from agarose gel by using a QIAquick gel extraction kit from QIAgen following the manufacturer's instructions (Chatsworth, Calif.), and digested with appropriate restriction endonucleases (Pharmacia Canada Inc, Baie d'Urfé, Canada). The pET vectors (Novagen, Madison, Wis.) were digested likewise and purified from agarose gel using a QIAquick gel extraction kit from QIAgen (Chatsworth, Calif.). The digested PCR products were ligated to the enzyme restricted pET expression vector. The ligated products were transformed into E. coli strain DH5α [φ80dlacZΔM15 Δ(lacZYA-argF)U169 endA1 recA1 hsdR17(r_{K}−m_{K}+) deoR thi-1 supE44 λ⁻gyrA96 relA1] (Gibco BRL, Gaithersburg, Md.) according to the method of Simanis (Hanahan D. (1985) In: DNA Cloning. D. M. Glover, ed., IRL Press, Washington D.C. vol. 1 pp. 109-135). Recombinant plasmid (rpET) containing genes were purified using a QIAgen kit (Chatsworth, Calif.) and DNA inserts were sequenced (Taq Dye Deoxy Terminator Cycle Sequencing kit, ABI, Foster City, Calif.).

TABLE 1
List of oligonucleotide primers for DNA cloning.

| PCR Primer set | | | | | |
|---|---|---|---|---|---|
| | | | | Nu- | |
| | | | | cleo- | |
| | | tide | | | |
| | | | | posi- | |
| | | | Re- | tion | |
| | | | stric- | (Seq. | Cloning |
| | Seq. | | tion | ID | vector- |
| Name | ID. | Sequence 5′-3′ | sites | No) a | Strain |
| COUF29 | 39 | CTCTCCGTCCATATGT | NdeI | 61- | pET19-b+ |
| | | GTGACGTACGGTCTAA | | 777 | |
| | | GG | | (1) | |
| COUF31 | 40 | TCAGGAGGATCCACTT | BamHI | | AD494 |
| | | ACTTAGTCATTCACCT | | | (pLysS) |
| | | TGATTTCCTTCTTG | | | |
| COUF75 | 41 | CACAACGTGTTCATAT | NdeI | 1-741 | pET19-b+ |
| | | GAAAATCACCACAGTC | | (3) | |
| | | AAAC | | | |
| | | CTATCCGGATCCACTT | BamHI | | AD494 |
| COUF76 | 42 | ACTTAGTCAGGAGGTT | | | (pLysS) |
| | | GGTGCGGGAG | | | |
| COUF110 | 43 | GGGAATTCCATATGCT | NdeI | 1-1584 | pET19-b+ |
| | | CCGTTTCTTCGCTG | | (5) | |
| COUF96 | 44 | GGTCCTCGAGTTACTT | XhoI | | Tuner |
| | | AGCTATAGTTTTTTCT | | | (DE3) |
| | | ATAAAACG | | | |
| COUF77 | 45 | CCGCTACCGCCATATG | NdeI | 1-1173 | pET19-b+ |
| | | TCATCTCCTGT | | (7) | |
| | | GCCCGCTCGAGCTACT | XhoI | | BL21 |
| COUF78 | 46 | TACTTACTGACCATCT | | | (DE3) |
| | | CCTGT | | | |
| COUF160 | 47 | CTCTATAGGTCCATAT | NdeI | 1-1977 | pET21-b+ |
| | | GAAACTACTTCTGAAA | | (9) | |
| | | GCG | | | |
| | | GATCCCTCGAGAGTTG XhoI | | | |
| COUF161 | 48 | TTATTGCCATGTC | | | BL21 |
| | | | | | (DE3) |
| COUF104 | 49 | CATGGGGGACATATGG | NdeI | 1-450 | pET19-b+ |
| | | CTGTTCAATCTAT | | (11) | |
| COUF105 | 50 | GTCGCTCGAGTACTTA | XhoI | | AD494 |
| | | CCTAACTTCCCGCCCC | | | (pLysS) |
| | | TG | | | |
| COUF102 | 51 | GTTGGAATTCATATGA | NdeI | 1-486 | pET19-b+ |
| | | GCAGTTCGGAAGTTG | | (13) | |
| COUF103 | 52 | GGGCCTCGAGTTACTC | XhoI | | BL21 |
| | | AATCATCTGACATCTG | | | (DE3) |
| | | | | | (pLysS) |
| COUF207 | 53 | GTAGTTAATCCATGGG | NcoI | 1-1168 | PET21-d+ |
| | | AACTCCTATATCTGG | | (15) | |
| COUF208 | 54 | GTCCTGACTCGAGGTC | XhoI | | BL21 |
| | | TTTGGGCTCTATTGAT | | | (DE3) |
| | | G | | | |
| COUF99 | 55 | GGCTACGCGCATATGC | NdeT | 1-1035 | pET19-b+ |
| | | TCACCCTAGGCTTGG | | (17) | |
| COUF123 | 56 | GCGCGGATCCTCACGC | BamHI | | BL21 |
| | | TACGGAGAGGCTAAGG | | | (DE3) |
| | | AG | | | |
| COUF79 | 57 | CTCGGCTGCTGCCATA | NdeI | 1-1249 | pET19-b+ |
| | | TGGGGCTGCAATCCAG | | (19) | |
| | | G | | | |
| COUF80 | 58 | GCCCGCTCGAGCTACT | XhoI | | BL21 |
| | | TACTTAATTCATCTTC | | | (DE3) |
| | | GTAAAGAATC | | | |
| COUF108 | 59 | CGCGAGAATTCATATG | NdeI | 1-1149 | pET19-b+ |
| | | GGAATCAATCCTTCG | | (21) | |
| COUF109 | 60 | GGCGCTCGAGTTATTC | XhoI | | BL21 |
| | | AAAAAGATCCCCG | | | (DE3) |
| COUF63 | 61 | GGAAGCATGCCATGGC | NcoI | 1-1119 | pFT3O-b+ |
| | | AATAGCTATTGCAAGG | | (23) | |
| | | G | | | |
| COUF65 | 62 | CATTTTTGGGGATCCA | BamHI | | BL21 |
| | | CTTACCTTAGCTAATT | | | (DE3) |
| | | ATCGAAATGTC | | | (pLysS) |
| COUP209 | 63 | CTATTAATAAAATCAT | NdeI | 1-1213 | pET21-b+ |
| | | ATGACAGATTCTAATC | | (25) | |
| | | CCCTAC | | | |
| | | GCTTTCCGACATCTCG | XhoI | | BL21 |
| COUF210 | 64 | AGGGGAGTCGTTAGAT | | | (DE3) |
| | | CGAAACG | | | |
| COUP211 | 65 | ATTTTAGAGGTCCATG | NcoI | 1-960 | pET21-d+ |
| | | GTGCGGCTCTTATCTA | | (27) | |
| | | TAC | | | |
| | | CTACGTAGGACTCGAG | XhoI | | BL21 |
| COUF212 | 66 | ACTCCCTAAAGATTTA | | | (DE3) |
| | | GATTC | | | |
| COUF213 | 67 | GTATTAAATGAAACCA | NdeI | 1-3255 | pET21-b+ |
| | | TATGGCAATGGATTTC | | (29) | |
| | | AACCCAG | | | |
| | | CATAGAGTCCCTCGAG | XhoI | | |
| COUF214 | 68 | TGAAGCGTTGATTAAG | | | BL21 |
| | | AAGC | | | (DE3) |
| COUF215 | 69 | CATTAACATTAAACAT | NdeI | 73- | pET21-b+ |
| | | ATGTTCGTAATGAAAA | | 1944 | |
| | | AACTTGTCC | | (31) | |
| | | | XhoI | | |
| COUF216 | 70 | GACCAACAGCTCGAGT | | | BL21 |
| | | TTTCTACACTGTTGTA | | | (DE3) |
| | | AT | | | |
| COUF2l7 | 71 | CTTAAGAGGGACATAT | NdeI | 1-1320 | pET21-b+ |
| | | GTCTPAGATAGTTTAT | | (33) | |
| | | AAATTTG | | | |
| | | GTAAATGCATGATGCG | NotI | | BL21 |
| COUF2l8 | 72 | GCCGCCGTGGCGACTA | | | (DE3) |
| | | TAGC | | | |
| COUF219 | 73 | CTTTAGGTAATTCATA | NdeI | 1-930 | pET21-b+ |
| | | TGAGAAAACTTATTTT | | (35) | |
| | | ATGC | | | |
| | | GTAAGCCTAAACTCGA | XhoI | | BL2l |
| COUP22O | 74 | GAGAACAACGGAGTTC | | | (DE3) |
| | | TTTTG | | | |
| COUF221 | 75 | GCCTCCGTAGCTCATA | NdeI | 91- | pET21-b+ |
| | | TGGTTATGGGAACCTA | | 879 | |
| | | TATCG | (37) | | |
| | | GCGCTTCCCCTCGAGT | XhoI | | BL21 |
| C0UF222 | 76 | ACATTTGTATTGATTT | | | (DE3) |
| | | CAG | | | |

^{a}Nucleotide position of DNA fragment amplified by PCR from gene (SEQ ID No) used as template.

### EXAMPLE 2

This example describes the PCR amplification and sequencing of BVH-CPN1 gene from other C. pneumoniae strains and the evaluation of the level of molecular conservation of this gene.

C. pneumoniae CWL-029 and CM-1 isolates were provided by the American Culture Collection (Rockville, Md.); C. pneumoniae TW-183 and AR39 isolates were provided by the Washington Research Foundation (Seattle, Wash.); C. pneumoniae strains YK-41 and KKpn-1 were provided by Akira Matsumoto (okayama,japan); C. pneumoniae strains WI-02 to WI-06 were provided by The Center of Disease Control (Atlanta, Ga.) ; C. pneumoniae strain UZG-1 was provided by Jacobus M. Ossewaarde (Bilthoven, The Netherlands). The respective coding region of C. pneumoniae gene BVH-CPN1 from these strains were amplified by PCR (DNA Thermal Cycler GeneAmp PCR system 2400 Perkin Elmer, San Jose, Calif.) from genomic DNA using the following oligos: COUF29 (5′-CTCTCCGTCCATATGTGTGACGTACGGTCTAAGG-3′); COUF31 (5′-TCAGGAGGATCCACTTACTTAGTCATTCACCTTGATTTCCTTCTTG -3′). PCR products were purified from agarose gel using a QIAquick gel extraction kit from QIAgen following the manufacturer's instructions (Chatsworth, Calif.) and the DNA insert were sequenced (Taq Dye Deoxy Terminator Cycle Sequencing kit, ABI, Foster City, Calif.) . Length of PCR fragments generated for all these strains was identical and pairwise comparisons of the nucleotides and the predicted amino acid sequences from four of these strains (CWL-029, KKpn-1, Wi-04 and UZG-1) revealed 100% identity. (PCR fragments of other strains have not been sequenced).

### EXAMPLE 3

This example illustrates the production and purification of recombinant C. pneumoniae BVH-CPN1 polypeptide.

The recombinant pET19 plasmid with BVH-CPN1 gene corresponding to the SEQ ID NO: 1 was used to transform by electroporation (Gene Pulser II apparatus, BIO-RAD Labs, Mississauga, Canada) E. coli strain AD494 (DE3) pLysS [Δara-leu7697 ΔlacX74 ΔphoA PvuII phoR ΔmalF3 F′[lac⁺(lacI¹) pro] trxB::Kan (DE3) pLysS(CM^{r})] (Novagen, Madison, Wis.). In this strain of E. coli, the T7 promotor controlling expression of the recombinant protein is specifically recognized by the T7 RNA polymerase (present on the λDE3 prophage) whose gene is under the control of the lac promotor which is inducible by isopropyl-β-d-thio-galactopyranoside (IPTG). The transformant AD494(DE3)pLysS/rpET19 was grown at 37° C. with agitation at 250 rpm in LB broth (peptone 10 g/L, yeast extract 5 g/L, NaCl 10 g/L) containing 100 μg/ml of carbenicillin (Sigma-Aldrich Canada Ltd., Oakville, Canada), 15 μg/ml of kanamycin (Sigma) and 30 μg/ml of chloramphenicol (Sigma) until the A₆₀₀ reached a value of 0.5. In order to induce the production of C. pneumoniae BVH-His●Tag-CPN1 recombinant protein, the cells were incubated for 3 additional hours at 30° C. in presence of IPTG at a final concentration of 1 mM. Induced cells from a 500 mL culture were pelleted by centrifugation and frozen at −70° C.

The purification of the recombinant proteins from the soluble cytoplasmic fraction of IPTG-induced AD494(DE3)pLysS/rpET19 was done by affinity chromatography based on the properties of the His●Tag sequence (10 consecutive histidine residues) to bind to divalent cations (Ni²⁺) immobilized on the His●Bind metal chelation resin. Briefly, the pelleted cells obtained from a 500 mL culture induced with IPTG was resuspended in lysis buffer (20 mM Tris, 500 mM NaCl, 5 mM imidazole, pH 7.9) containing 1 mM of (Phenylmethylsulfonyl fluoride (PMSF; Sigma), sonicated and centrifuged at 16,000×g for 30 min to remove debris. The supernatant was deposited on a Ni-NTA agarose column (QIAgen, Mississauga, Ontario, Canada). The C. pneumoniae BVH-His●Tag-CPN1 recombinant protein was eluted with 500 mM imidazole-500 mM NaCl-20 mM Tris pH 7.9. The removal of the salt and imidazole from the sample was done by dialysis against PBS at 4° C. The quantity of recombinant protein obtained from the soluble fraction of E. coli was estimated by MicroBCA (Pierce, Rockford, Ill.).

### EXAMPLE 4

This example illustrates the analysis of the immunogenicity and the protective ability of mice against C. pneumoniae infection following immunization with BVH-CPN1.

Groups of 7 female BALB/c mice (Charles River, Ontario, Canada) were immunized intranasally four times at two-week intervals with 20 μg of affinity purified C. pneumoniae His●Tag recombinant protein in presence of choleric toxin (CT; 1 μg) adjuvant or the E. coli heat labile toxin (LT; 1 μg) adjuvant (Cedarlane Laboratories Ltd, Hornby, Canada) or, as control, with adjuvant alone in PBS. Subcutaneous immunizations were done also in presence of QuilA (15 μg) (Cedarlane Laboratories Ltd, Hornby, Canada),three times at two-week intervals. Blood samples were collected from the orbital sinus on day prior to each immunization and 14 days following the third (day 42) or fourth (day 56) injection. Antibody titers were determined by microimmunofluorescence on confluent HEp-2 monolayers cells cultured into microculture plates (96 wells) and infected with 0.5 inclusion-forming unit (IFU) of C. pneumoniae CWL-029 per cell. The secondary antibody used was a goat anti-mouse IgG+IgM (H+L) (Fc specific) conjugated to fluorescein isothiocyanate (FITC) (Jackson Immunoresearch Labs, Mississauga, Ontario). The reactive titer of an antiserum was defined as the reciprocal of the smaller dilution showing fluorescent signal. To investigate whether these immune responses elicited by BVH-CPN1 was functionally significant, in vivo protective efficacy was evaluated 28 days later in mice challenged intranasally with approximately 2×10⁶ IFU of the C. pneumoniae strain CWL-029. To measure the effectiveness of vaccination to limit the in vivo growth of Chlamydia, 5 days following the lung infection bacterial, the bacterial clearance is provided on intact lungs following excision and homogenization in a tissue homogeniser in presence of conservation buffer (7.5% sucrose-3.8 mM KH₂PO₄-7mM K₂HPO₄-5mM glutamic acid). Serial dilutions of supernatants of these lung homogenates were plated on HEp-2 monolayers cells (Epermidoid carcinoma from human larynx; American Type Culture Collection, Rockville, Md.) for IFU determination. Mice injected with adjuvant only were used as negative controls.

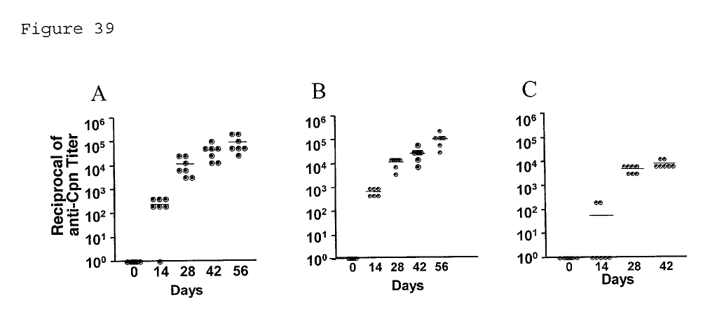

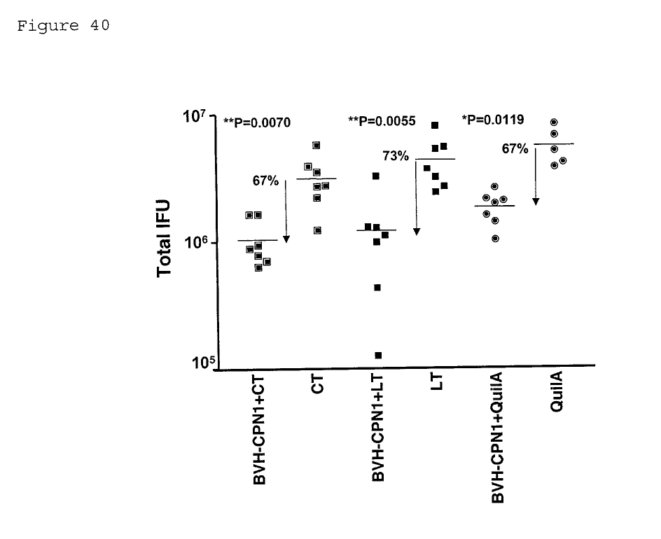

The results of immunogenicity study are illustrated in FIG. 39. As shown in FIG. 39, the final bleed sera obtained from mice intranasally immunized with BVH-CPN1 in presence of CT or LT are equivalent. By comparison, subcutaneous immunization in presence of QuilA gave a 10 fold lower titer following the third immunization. However, titer variations did not seem to have an effect on protective ability of mice as shown in FIG. 40. Mice, intranasally or subcutaneously immunized with BVH-CPN1 had chlamydial lung titers that were over 3 to 3.6-fold lower than those of control mice immunized with adjuvant only, giving a protection level ranged from 67 to 73%.

### EXAMPLE 5

This example illustrates the accessibility to antibodies of the BVH-CPN1 protein at the surface of C. pneumoniae strain CWL-029.

A pellet of purified chlamydiae (1×10⁸ IFU) was resuspended in 500 μl of polyvalent sera elicited by mice immunized with the BVH-CPN1 polypeptide. The suspension was incubated while shaking for 6 hours at 4° C. After two washes with PBS, the chlamydiae were incubated for 6 hours at 4° C. with a goat anti-mouse IgG gold conjugated (12 nm) antibody (Jackson Immunoresearch Labs, Mississauga, Ontario). After two washes with ammonium acetate (0.1 M), the pellet was resuspended in 50 μL of ammonium acetate (0.1M). Chlamydiae were stained with uranyl acetate (2%) and viewed in an electron microscope (Phillips EM 300, Phillips, Germany) at 60 kV. Micrographs were taken at a magnification of ×92 400 and ×148 500.

The electron micrographs of chlamydiae preparations showed the immunogold labelling of surface antigens on the reticulate forms. No gold particles were seen to be associated with chlamydiae that had been reacted with preimmune sera.

What is claimed is:

1. An isolated polynucleotide comprising a polynucleotide chosen from:
(a) a polynucleotide encoding a polypeptide having at least 70% identity to a second polypeptide comprising a sequence chosen from: SEQ ID NOs: 2, 4, 6, 8, 10, 12, 14, 16, 18, 20, 22, 24, 26, 28, 30, 32, 34, 36 and 38 or fragments or analogs thereof;
(b) a polynucleotide encoding a polypeptide having at least 95% identity to a second polypeptide comprising a sequence chosen from: SEQ ID NOs: 2, 4, 6, 8, 10, 12, 14, 16, 18, 20, 22, 24, 26, 28, 30, 32, 34, 36 and 38 or fragments or analogs thereof;
(c) a polynucleotide encoding a polypeptide comprising a sequence chosen from: SEQ ID NOs: 2, 4, 6, 8, 10, 12, 14, 16, 18, 20, 22, 24, 26, 28, 30, 32, 34, 36 and 38 or fragments or analogs thereof;
(d) a polynucleotide encoding a polypeptide capable of generating antibodies having binding specificity for a polypeptide having a sequence chosen from SEQ ID NOs: 2, 4, 6, 8, 10, 12, 14, 16, 18, 20, 22, 24, 26, 28, 30, 32, 34, 36 and 38 or fragments or analogs thereof;
(e) a polynucleotide encoding an epitope bearing portion of a polypeptide having a sequence chosen from: SEQ ID NOs: 2, 4, 6, 8, 10, 12, 14, 16, 18, 20, 22, 24, 26, 28, 30, 32, 34, 36 and 38 or fragments or analogs thereof;
(f) a polynucleotide that is complementary to a polynucleotide in (a), (b), (c), (d) or (e).

2. An isolated polynucleotide comprising a polynucleotide chosen from:
(a) a polynucleotide encoding a polypeptide having at least 70% identity to a second polypeptide comprising a sequence chosen from: SEQ ID NOs: 2, 4, 6, 8, 10, 12, 14, 16, 18, 20, 22, 24, 26, 28, 30, 32, 34, 36 and 38;
(b) a polynucleotide encoding a polypeptide having at least 95% identity to a second polypeptide comprising a sequence chosen from: SEQ ID NOs: 2, 4, 6, 8, 10, 12, 14, 16, 18, 20, 22, 24, 26, 28, 30, 32, 34, 36 and 38;
(c) a polynucleotide encoding a polypeptide comprising a sequence chosen from: SEQ ID NOs: 2, 4, 6, 8, 10, 12, 14, 16, 18, 20, 22, 24, 26, 28, 30, 32, 34, 36 and 38;
(d) a polynucleotide encoding a polypeptide capable of generating antibodies having binding specificity for a polypeptide having a sequence chosen from SEQ ID NOs: 2, 4, 6, 8, 10, 12, 14, 16, 18, 20, 22, 24, 26, 28, 30, 32, 34, 36 and 38;
(e) a polynucleotide encoding an epitope bearing portion of a polypeptide having a sequence chosen from: SEQ ID NOs: 2, 4, 6, 8, 10, 12, 14, 16, 18, 20, 22, 24, 26, 28, 30, 32, 34, 36 and 38;
(f) a polynucleotide that is complementary to a polynucleotide in (a), (b), (c), (d) or (e).

3. The polynucleotide of claim 1, wherein said polynucleotide is DNA.

4. The polynucleotide of claim 2, wherein said polynucleotide is DNA.

5. The polynucleotide of claim 1, wherein said polynucleotide is DNA.

6. The polynucleotide of claim 2, wherein said polynucleotide is DNA. RNA.

7. The polynucleotide of claim 1 that hybridizes under stringent conditions to either
(a) a DNA sequence encoding a polypeptide or
(b) the complement of a DNA sequence encoding a polypeptide;
wherein said polypeptide comprises SEQ ID NO: 2, 4, 6, 8, 10, 12, 14, 16, 18, 20, 22, 24, 26, 28, 30, 32, 34, 36, 38 or fragments or analogs thereof.

8. The polynucleotide of claim 1 that hybridizes under stringent conditions to either
(a) a DNA sequence encoding a polypeptide or
(b) the complement of a DNA sequence encoding a polypeptide;
wherein said polypeptide comprises SEQ ID NO: 2, 4, 6, 8, 10, 12, 14, 16, 18, 20, 22, 24, 26, 28, 30, 32, 34, 36, 38.

9. The polynucleotide of claim 1 that hybridizes under stringent conditions to either
(a) a DNA sequence encoding a polypeptide or
(b) the complement of a DNA sequence encoding a polypeptide;
wherein said polypeptide comprises at least 10 contiguous amino acid residues from a polypeptide comprising SEQ ID NO: 2, 4, 6, 8, 10, 12, 14, 16, 18, 20, 22, 24, 26, 28, 30, 32, 34, 36, 38 or fragments or analogs thereof.

10. The polynucleotide of claim 1 that hybridizes under stringent conditions to either
(a) a DNA sequence encoding a polypeptide or
(b) the complement of a DNA sequence encoding a polypeptide;
wherein said polypeptide comprises at least 10 contiguous amino acid residues from a polypeptide comprising SEQ ID NO: 2, 4, 6, 8, 10, 12, 14, 16, 18, 20, 22, 24, 26, 28, 30, 32, 34, 36, 38.

11. A vector comprising the polynucleotide of claim 1, wherein said DNA is operably linked to an expression control region.

12. A vector comprising the polynucleotide of claim 2, wherein said DNA is operably linked to an expression control region.

13. A host cell transfected with the vector of claim 11.

14. A host cell transfected with the vector of claim 12.

15. A process for producing a polypeptide comprising culturing a host cell according to claim 13 under conditions suitable for expression of said polypeptide.

16. A process for producing a polypeptide comprising culturing a host cell according to claim 14 under conditions suitable for expression of said polypeptide.

17. An isolated polypeptide comprising a member chosen from:
(a) a polypeptide having at least 70% identity to a second polypeptide having an amino acid sequence chosen from: SEQ ID NOs: 2, 4, 6, 8, 10, 12, 14, 16, 18, 20, 22, 24, 26, 28, 30, 32, 34, 36, 38 or fragments or analogs thereof;
(b) a polypeptide having at least 95% identity to a second polypeptide having an amino acid sequence chosen from: SEQ ID NOs: 2, 4, 6, 8, 10, 12, 14, 16, 18, 20, 22, 24, 26, 28, 30, 32, 34, 36, 38 or fragments or analogs thereof;
(c) a polypeptide comprising a sequence chosen from: SEQ ID NOs: 2, 4, 6, 8, 10, 12, 14, 16, 18, 20, 22, 24, 26, 28, 30, 32, 34, 36, 38 or fragments or analogs thereof;
(d) a polypeptide capable of generating antibodies having binding specificity for a polypeptide having a sequence chosen from SEQ ID NOs: 2, 4, 6, 8, 10, 12, 14, 16, 18, 20, 22, 24, 26, 28, 30, 32, 34, 36, 38 or fragments or analogs thereof;
(e) an epitope bearing portion of a polypeptide having a sequence chosen from SEQ ID NOs: 2, 4, 6, 8, 10, 12, 14, 16, 18, 20, 22, 24, 26, 28, 30, 32, 34, 36, 38 or fragments or analogs thereof;
(f) the polypeptide of (a), (b), (c), (d) or (e) wherein the N-terminal Met residue is deleted;
(g) the polypeptide of (a), (b), (c), (d) or (e) wherein the secretory amino acid sequence is deleted.

18. An isolated polypeptide comprising a member chosen from:
(a) a polypeptide having at least 70% identity to a second polypeptide having an amino acid sequence chosen from: SEQ ID NOs: 2, 4, 6, 8, 10, 12, 14, 16, 18, 20, 22, 24, 26, 28, 30, 32, 34, 36, 38;
(b) a polypeptide having at least 95% identity to a second polypeptide having an amino acid sequence chosen from: SEQ ID NOs: 2, 4, 6, 8, 10, 12, 14, 16, 18, 20, 22, 24, 26, 28, 30, 32, 34, 36, 38;
(c) a polypeptide comprising a sequence chosen from: SEQ ID NOs: 2, 4, 6, 8, 10, 12, 14, 16, 18, 20, 22, 24, 26, 28, 30, 32, 34, 36, 38;
(d) a polypeptide capable of generating antibodies having binding specificity for a polypeptide having a sequence chosen from SEQ ID NOs: 2, 4, 6, 8, 10, 12, 14, 16, 18, 20, 22, 24, 26, 28, 30, 32, 34, 36, 38;
(e) an epitope bearing portion of a polypeptide having a sequence chosen from SEQ ID NOs: 2, 4, 6, 8, 10, 12, 14, 16, 18, 20, 22, 24, 26, 28, 30, 32, 34, 36, 38;
(f) the polypeptide of (a), (b), (c), (d) or (e) wherein the N-terminal Met residue is deleted;
(g) the polypeptide of (a), (b), (c), (d) or (e) wherein the secretory amino acid sequence is deleted.

19. A chimeric polypeptide comprising two or more polypeptides having a sequence chosen from SEQ ID NOs: 2, 4, 6, 8, 10, 12, 14, 16, 18, 20, 22, 24, 26, 28, 30, 32, 34, 36, 38 or fragments or analogs thereof; provided that the polypeptides are linked as to formed a chimeric polypeptide.

20. A chimeric polypeptide comprising two or more polypeptides having a sequence chosen from SEQ ID NOs: 2, 4, 6, 8, 10, 12, 14, 16, 18, 20, 22, 24, 26, 28, 30, 32, 34, 36, 38 or fragments or analogs thereof; provided that the polypeptides are linked as to formed a chimeric polypeptide.

21. A pharmaceutical composition comprising a polypeptide according to any one of claims 17 to 20 and a pharmaceutically acceptable carrier, diluent or adjuvant.

22. A method for prophylactic or therapeutic treatment of Chlamydial bacterial infection in a host susceptible to Chlamydiae infection comprising administering to said host a therapeutic or prophylactic amount of a composition according to claim 21.

23. A method according to claim 22 wherein the host is an animal.

24. A method according to claim 22 wherein the host is a human.

25. A method according to claim 22 wherein said bacterial infection is caused by Chlamydia pneumoniae.

26. A method according to claim 22 wherein said bacterial infection is caused by Chlamydia psittaci.

27. A method according to claim 22 wherein said bacterial infection is caused by Chlamydia trachomatis.

28. A method according to claim 22 wherein said infection causes sinusitis, pharyngitis, bronchitis, pneumonitis, asthmatic bronchitis adult-onset asthma, chronic obstructive pulmonary diseases (CDPD), atherogenesis or atherosclerosis.

29. A method for diagnostic of chlamydial bacterial infection in a host susceptible to chlamydial infection comprising administering to said host the composition of claim 21.

30. A method for diagnostic of Chlamydia infection in a host susceptible to Chlamydia infection comprising
(a) obtaining a biological sample from a host;
(b) incubating an antibody or fragment thereof reactive with a Chlamydia polypeptide of any of the claims 17 to 20 with the biological sample to form a mixture; and
(c) detecting specifically bound antibody or bound fragment in the mixture which indicates the presence of Chlamydia.

31. A method for diagnostic of Chlamydia infection in a host susceptible to Chlamydia infection comprising
(a) obtaining a biological sample from a host;
(b) incubating one or more Chlamydia polypeptides of any of the claims 17 to 20 or fragments thereof with the biological sample to form a mixture; and
(c) detecting specifically bound antigen or bound fragment in the mixture which indicates the presence of antibody specific to Chlamydia.

32. Use of pharmaceutical method according to claim 22 for the prophylactic or therapeutic treatment of Chlamydia bacterial infection in a host susceptible to Chlamydia infection comprising administering to said host a therapeutic or prophylactic amount of a composition according to claim 21.

33. Kit comprising a polypeptide according to any one of claims 17 to 20 for detection or diagnosis of Chlamydia infection.
